(19) United States
(12) Patent Application Publication
(10) Pub. No.: US 2025/0152057 A1
(43) Pub. Date: May 15, 2025

(54) SENSOR HAVING DUAL LACTATE-RESPONSIVE ACTIVE AREAS AND METHODS FOR DETERMINING VARIANCE

(71) Applicant: ABBOTT DIABETES CARE INC., Alameda, CA (US)

(72) Inventors: Ting Chen, Alameda, CA (US); Yudhajit Das, Fremont, CA (US); Junli Ou, Pleasanton, CA (US); Matthew Bates, New Hope, PA (US); Carlo Di Iulio, Witney (GB)

(73) Assignee: ABBOTT DIABETES CARE INC., Alameda, CA (US)

(21) Appl. No.: 18/821,757

(22) Filed: Aug. 30, 2024

Related U.S. Application Data
(60) Provisional application No. 63/580,258, filed on Sep. 1, 2023.

(51) Int. Cl. A61B 5/1486; A61B 5/00; A61B 5/145

(52) U.S. Cl. CPC A61B 5/14865; A61B 5/1451; A61B 5/14546; A61B 5/7246

(57) ABSTRACT

The present disclosure describes lactate-responsive sensors having first and second lactate-responsive sensing areas, sensing systems incorporating the lactate-responsive sensor, and methods of using the same that for continuously monitoring lactate levels and determining variance between lactate concentrations derived from signals independently obtained from the first and second lactate-responsive areas.

### BACKGROUND

A typical implantable sensor is designed with a single channel for data collection. However, during in vivo use, many different factors can lead to erroneous sensor results. For example, insertion trauma and bleeding can cause sensor measurements to be much higher or lower than the actual analyte concentration and sensor movement during wear can lead to sensor signal variation as well.

In addition, early signal variation (ESV) and late signal variation (LSV) have been observed in current sensors for continuous analyte monitoring. For example, for lactate sensors, the signals indicative of lactate level can rise during the early and late wear days (e.g., the first two days and last 7 days of a sensor wear period).

Thus, there is a need for a sensor that provides continuous lactate monitoring without the inaccuracies associated with current sensors so that lactate levels can be tracked in real time.

### BRIEF SUMMARY

The present disclosure describes an analyte-responsive sensor having two analyte-responsive sensing areas (two channels) for detecting the same analyte, and sensing systems incorporating the analyte-responsive sensor. The present disclosure also describes methods for monitoring analyte levels and detecting variance between the channels so that the sensing system can report accurate analyte concentration data from at least one of the channels to a user.

In some embodiments, the present disclosure describes a lactate-responsive sensor comprising two lactate-responsive sensing areas, and sensing systems incorporating the lactate-responsive sensor. In some embodiments, the present disclosure also describes methods for monitoring lactate levels and detecting variance between the channels so that the sensing system can report accurate lactate concentration data from at least one of the channels to a user.

In some embodiments, the lactate responsive sensor comprises: a substrate; a first working electrode located on the substrate; a second working electrode located on the substrate; a first analyte-responsive sensing area disposed on a surface of the first working electrode; a second analyte-responsive sensing area disposed on a surface of the second working electrode; and a membrane that is permeable to the analyte overcoating the first and second analyte-responsive sensing areas, wherein the first and second analyte-responsive sensing areas are configured to detect the same analyte, and wherein the first and second analyte-responsive sensing areas are configured to independently produce first and second signals indicative of analyte concentrations measured at the first and second working electrodes, and wherein the sensor is configured to be partially inserted into a user's skin.

The present disclosure also relates to an analyte sensor comprising: a substrate; a first working electrode located on the substrate; a second working electrode located on the substrate; a first lactate-responsive sensing area disposed on a surface of the first working electrode; a second lactate-responsive sensing area disposed on a surface of the second working electrode; and a membrane that is permeable to lactate overcoating the first and second lactate-responsive sensing areas, wherein the first and second lactate-responsive sensing areas are configured to independently produce first and second signals indicative of lactate concentrations measured at the first and second working electrodes, and wherein the sensor is configured to be partially inserted into a user's skin.

In some embodiments, the first lactate-responsive sensing area comprises lactate oxidase.

In some embodiments, the first lactate-responsive sensing area can comprise a first polymer and a first electron transfer agent.

In some embodiments, the first electron transfer agent can be covalently bonded to the first polymer.

In some embodiments, the second lactate-responsive sensing area can comprise lactate oxidase.

In some embodiments, the second lactate-responsive sensing area can comprise a second polymer and a second electron transfer agent.

In some embodiments, the second electron transfer agent can be covalently bonded to the second polymer.

In some embodiments, the sensor further can comprise a reference electrode and a counter electrode.

In some embodiments, the sensor can be configured for insertion into a tissue.

In some embodiments, the membrane can comprise at least a crosslinked polyvinylpyridine homopolymer or copolymer.

The present disclosure also relates to a method for monitoring lactate levels in a user, comprising:
a) exposing an analyte sensor of a sensing system to a fluid; wherein the analyte sensor comprises
   a substrate;
   a first working electrode located on the substrate;
   a second working electrode located on the substrate;
   a first lactate-responsive sensing area disposed on a surface of the first working electrode;
   a second lactate-responsive sensing area disposed on a surface of the second working electrode; and
   a membrane that is permeable to lactate overcoating the first and second lactate-responsive sensing areas,
   wherein the first and second lactate-responsive sensing areas are configured to independently produce first and second signals indicative of lactate concentrations measured at the first and second working electrodes, and
   wherein the sensor is configured to be partially inserted into the user's skin;
b) applying a potential to the first working electrode and applying a potential to the second working electrode of the analyte sensor;
c) obtaining a first signal at or above an oxidation-reduction potential of the first lactate-responsive sensing area, the first signal being proportional to a concentration of lactate in the fluid;
d) obtaining a second signal at or above an oxidation-reduction potential of the second lactate-responsive sensing area, the second signal being proportional to a concentration of lactate in the fluid; and
e) correlating the first and second signals to first and second lactate concentrations in the fluid.

In some embodiments, the method further can comprise determining a variance between the first and second lactate concentrations.

In some embodiments, the first lactate-responsive sensing area can comprise lactate oxidase.

In some embodiments, the first lactate-responsive sensing area can comprise a first polymer and a first electron transfer agent.

In some embodiments, the first electron transfer agent is covalently bonded to the first polymer.

In some embodiments, the second lactate-responsive sensing area can comprise lactate oxidase.

In some embodiments, the second lactate-responsive sensing area can comprise a second polymer and a second electron transfer agent.

In some embodiments, the second electron transfer agent can be covalently bonded to the second polymer.

In some embodiments, the sensor further can comprise a reference electrode and a counter electrode.

In some embodiments, the sensor can be configured for insertion into a tissue.

In some embodiments, the membrane can comprise at least a crosslinked polyvinylpyridine homopolymer or copolymer.

The present disclosure also relates to a method comprising:
a) continuously measuring signals indicative of lactate concentrations in a biological fluid with a sensing system comprising a lactate-responsive sensor, the lactate-responsive sensor comprising: a substrate; a first working electrode located on the substrate; a second working electrode located on the substrate; a first lactate-responsive sensing area disposed on a surface of the first working electrode; a second-responsive sensing area disposed on a surface of the second working electrode; and a membrane that is permeable to lactate overcoating the first and second lactate-responsive sensing areas, wherein the first and second lactate-responsive sensing areas are configured to independently produce first and second signals indicative of lactate concentrations measured at the first and second working electrodes, and wherein the sensor is configured to be partially inserted into a tissue;
b) communicating first and second signals indicative of lactate concentrations measured at the first and second working electrodes to a processor;
c) correlating the first and second signals to corresponding first and second lactate concentrations; and
d) determining hybrid variance between the first and second lactate concentrations.

In some embodiments, determining the hybrid variance can comprise:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

In some embodiments, the method can further comprise transmitting an average of the first and second lactate concentrations to be displayed if the hybrid variance is less than or equal to about 40%.

In some embodiments, the method can further comprise detecting a noisy patch if a hybrid variance is greater than about 40% for about an hour or more.

In some embodiments, the method can further comprise calculating an absolute relative difference (ARD) between standard deviations of the first and second lactate concentrations for a period of time and transmitting an average of the first and second lactate concentrations for the period of time to be displayed when the ARD is less than or equal to about 40%.

In some embodiments, the method can further comprise calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

Additional embodiments and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 shows a diagram of an illustrative sensing system that can incorporate a lactate-responsive sensor of the present disclosure.

FIG. 2 shows a block diagram of a processing electronics that can be associated with one or more components of the sensing system.

FIG. 3A shows a cross-sectional diagram of illustrative two-electrode analyte sensor configuration having a single working electrode.

FIGS. 3B and 3C show diagrams of illustrative three-electrode analyte sensor configurations.

FIG. 4 shows a cross-section diagram of an analyte sensor having a first lactate-responsive sensing area and a second lactate-responsive sensing area upon separate working electrodes.

FIG. 5A shows a cross-sectional diagram of an illustrative analyte sensor configuration having two working electrodes, a counter electrode, and a reference electrode.

FIG. 5B shows a cross-sectional diagram of an illustrative analyte sensor configuration having two working electrodes, a counter electrode, and a reference electrode.

FIG. 6 shows an analyte sensor for detecting lactate according to some embodiments of the present disclosure.

FIG. 7 shows a diagram of an enzyme system that can be used for detecting lactate according to the disclosure herein.

FIG. 8 shows a flow chart for detecting and removing noisy patches in lactate measurements based on signals indicative of lactate concentrations from the first and second working electrodes of the sensor.

FIG. 9A shows a graph of exemplary lactate measurements based on signals from system comprising first and second working electrodes.

FIG. 9B shows a graph of the hybrid variance between the exemplarily lactate measurements from Channels 1 and 2, as show in FIG. 9A.

FIG. 10A shows a graph of exemplary lactate measurements based on signals from the first working electrode before and after noisy segments were detected and removed by the algorithm described herein.

FIG. 10B shows a graph of exemplary lactate measurements based on signals from the second working electrode before and after noisy segments were detected and removed by the algorithm.

### DETAILED DESCRIPTION

The headings provided herein are not limitations of the various embodiments of the disclosure, which can be defined by reference to the specification as a whole. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “measure” and variations thereof can encompass the meaning of a respective term, such as “determine,” “calculate,” and variations thereof.

As used herein, an “analyte” is an enzyme substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample via electrochemical oxidation and reduction reactions on the sensor. These reactions are transduced to an electrical signal that can be correlated to an amount, concentration, or level of an analyte in the sample.

As used herein, a “working electrode” is an electrode at which the analyte (or a second compound whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated, or unless a reference electrode is also present, in which case the term “counter electrode” is intended to refer solely to a counter electrode.

As used herein, the term “reference electrode” refers to an electrode whose potential is known and can be used as a reference against which the working electrode potential is assessed or measured. In the context of embodiments of the present disclosure, the term “reference electrode” includes both a) reference electrodes and b) reference electrodes that also function as counter electrodes (i.e., counter/reference electrodes), unless otherwise indicated, or unless a counter electrode is also present, in which case the term “reference electrode” is intended to refer only to a reference electrode.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

As used herein, a “sensing area” is a component of the sensor including constituents that facilitate the electrolysis of the analyte. The sensing area can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some embodiments of the present disclosure, a sensor includes a sensing area that is non-leachably disposed in proximity to or on the working electrode. In some embodiments of the present disclosure, the sensing area can be continuously or discontinuously disposed on the working electrode. A sensing area is considered to be “continuously disposed” on the working electrode when the sensing area is applied in a fashion that is uninterrupted across the surface of the working electrode, i.e. a single spot or line, etc. A sensing area is considered to be “discontinuously disposed” on the working electrode when the sensing area is applied as at least two discrete shapes on the working electrode, such as two spots, two lines, a spot and a line, or a plurality (e.g., an array) of spots, lines, or combination thereof. The number of discontinuous applications of the sensing area as a series of spots and/or lines is not considered to be particularly limited, but can range from 2 to about 10 (e.g., 2, 3, 4, 5, 6, 7, 8, 9, or 10, including about 3 to about 8, or from about 4 to about 6). In some embodiments, the sensing area is continuously disposed on the working electrode. In some embodiments, the sensing area is discontinuously disposed on the working electrode.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

The term “patient” refers to a living animal, and thus encompasses a living mammal and a living human, for example. The term “user” can be used herein as a term that encompasses the term “patient.”

As used herein, the term “simultaneous” and related terms such as “simultaneously,” refer to two or more actions that take place at exactly the same time or at substantially the same time. Two actions take place at substantially the same time when the actions overlap in time or are separated in time by a period of time that would be understood by a person of skill in the art to be de minimis under the circumstances, i.e. by microseconds, by seconds, etc.

### Sensors, Sensor Control Devices, and Sensing Systems

The present disclosure describes a lactate-responsive sensor and sensing systems incorporating a lactate-responsive sensor that are beneficial for monitoring lactate levels and detecting variance between the channels so that the sensing system can report accurate lactate concentration data from at least one of the channels to a user. In some embodiments, the system is a continuous lactate monitoring system.

In some embodiments, the sensor is configured to detect one or more lactate levels. In some embodiments, the sensor is configured to measure one or more lactate levels about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

In some embodiments, the sensor is configured to communicate a signal that is indicative of the one or more lactate levels to a processor. In some embodiments, the sensor can be a sensor as described in the present disclosure.

In some embodiments, the system can include various sensing components, such as a processor and/or coding instructions (algorithms) therein, that are adapted to process sensor data received from the lactate sensor and determine a plurality of lactate concentrations therefrom. The processor and/or coding instructions can then analyze the lactate concentrations to determine and remove abnormal signal variations.

In some embodiments, the system can comprise a lactate-responsive sensor configured to detect lactate in vivo, and a processor located in a cloud server, remote terminal or a local terminal that is communicatively coupled to the lactate-responsive sensor. Cloud- or server-based communication also falls within the scope of the systems disclosed herein. As used herein, the term “local terminal” refers to a user interface that is physically contiguous with a system where the lactate-responsive sensor is located. For example, in some embodiments, the processor can be contiguous with a housing of the lactate-responsive sensor. As used herein, the term “remote terminal” refers to a user interface that is not located in the same physical space where the lactate-responsive sensor is located. In some embodiments, the remote terminal and its processor can be communicatively coupled to the lactate-responsive sensor or a network. In some embodiments, an individual interfacing with the system can be blinded to the output of the lactate-responsive sensor. In other embodiments, an individual can see the sensor output (e.g., lactate concentrations) in real-time or near real-time, such as on a remote or local viewable display. Remote terminals can include, for example, a dedicated reader device, a dedicated fitness monitoring device (e.g., a FITBIT), a smart phone, or a smart watch.

In some embodiments, the processor can be configured to receive a signal from the lactate-responsive sensor. The processor can be further configured to determine a plurality of lactate concentrations upon receipt of the received signals from the sensor and determine and remove abnormal signal variations as disclosed below. The processor can further signal an individual wearing the sensor or another interested party when predetermined lactate levels have been reached, a specified lactate concentration, a multiple of a baseline lactate concentration, or a fraction of a peak lactate concentration, for example. The output of the processor can be numerical and/or graphical. The notification to the wearer of the lactate-responsive sensor or other interested party can be auditory, tactile (haptic), or any combination thereof.

In some embodiments, the active sensing region of the lactate-responsive sensor can be disposed in any suitable location in vivo. Suitable locations can include, but are not limited to, intravenous, subcutaneous, or dermal locations. An intravenous sensor can have the advantage of analyzing lactate directly in blood, but is invasive and can sometimes be painful for an individual to wear over an extended period. Subcutaneous and dermal analyte sensors can often be less painful for an individual to wear due to their shallower penetration and can provide sufficient measurement accuracy in many cases. In some embodiments, the lactate-responsive sensor suitable for use in the present disclosure can be a dermal sensor configured to interrogate dermal fluid of an individual. In some embodiments, the lactate-responsive sensor suitable for use in the present disclosure can be configured to interrogate interstitial fluid of an individual. As used herein, the term “interrogate” refers to the act of measuring a parameter of a sample.

In some embodiments, the sensor can extend from a housing that is configured for external wear upon the skin of an individual performing a given physical activity. The external location where the lactate-responsive sensor is placed is not considered to be particularly limited and can be dependent upon the type of physical activity being performed. In some embodiments, the lactate-responsive sensor can be placed upon the biceps, triceps, upper back, lower back, chest, buttocks, abdomen, thigh, or calf. In some embodiments, multiple lactate-responsive sensors can be used to monitor an individual, such as to perform as a comparison between lactate concentrations measured at two different external locations. If desired, the outputs from one or both sensor locations can also be cross-referenced with blood lactate readings obtained from finger or ear lobe pricks.

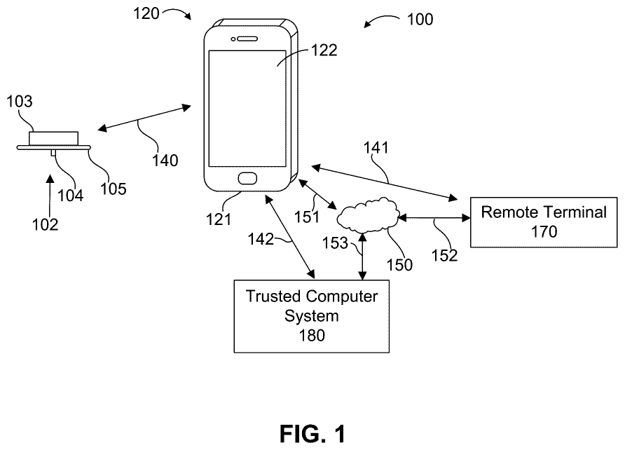

FIG. 1 shows a diagram of an illustrative system that can incorporate any lactate-responsive sensor of the present disclosure. As shown, system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing lactate concentrations and alerts or notifications determined by sensor 104 or a processor associated therewith, as well as allowing for one or more user inputs, according to some embodiments. Alternately, reader device 120 can produce output that is blinded to a user. Reader device 120 can be a multi-purpose smartphone or a dedicated electronic reader instrument. While only one reader device 120 is shown, multiple reader devices 120 can be present in certain instances. A suitable processor can also be incorporated in reader device 120, according to some embodiments. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer systems 180 without an intervening reader device 120 being present. For example, sensor 104 can communicate with remote terminal 170 and/or trusted computer system 180 through a direct communication link to network 150, according to some embodiments, as described in U.S. Patent Application Publication 2011/0213225 which is incorporated herein by reference in its entirety. Any suitable electronic communication protocol can be used for each communication path or link 141, 142, 151, 152 and/or 153, such as near field communication (NFC), radio frequency identification (RFID), BLUETOOTH® or BLUETOOTH® Low Energy protocols, WiFi, mobile telephone network, or the like. Remote terminal 170 and/or trusted computer system 180 can be accessible, according to some embodiments, by a party other than a primary user who have an interest in the primary user's lactate concentrations or rate of lactate clearance, such as the individual's trainer or coach. Reader device 120 can comprise display 122 and optional input component 121. Display 122 can comprise a touch-screen interface, according to some embodiments.

In some embodiments, sensor control device 102 can include sensor 104 that, while positioned in vivo, makes contact with the bodily fluid of the user and senses the analyte levels contained therein. The sensor can be part of the sensor control device that resides on the body of the user and contains the electronics and power supply that enable and control the analyte sensing. Sensor control device 102, and variations thereof, can also be referred to as a “sensor control unit,” an “on-body electronics” device or unit, an “on-body” device or unit, or a “sensor data communication” device or unit. In some embodiments, sensor control device 102 includes sensor housing 103, which can house circuitry and a power source for operating sensor 104. Optionally, the power source and/or active circuitry can be omitted. A processor (not shown in FIG. 1) can be communicatively coupled to sensor 104, with the processor being physically located within sensor housing 103 or reader device 120. In some embodiments, sensor 104 protrudes from the underside of sensor housing 103 and extends through adhesive layer 105, which is adapted for adhering sensor housing 103 to a tissue surface, such as skin, according to some embodiments.

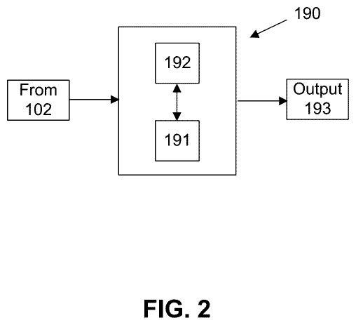

FIG. 2 shows a block diagram of a processing electronics that can be associated with one or more components of the sensing system, such as within reader device 120. Alternately, such functionality can be associated with one or more of network 150, remote terminal 170 or trusted computer system 180. As shown, processing electronics 190 receive, either directly or indirectly, a signal originating from sensor control device 102. The signal can be processed using algorithms associated with processor 191 and/or memory 192. Lactate concentrations determined therewith can be stored in memory 192 and/or exported to output device 193, which can be a display or external storage medium in various embodiments. Guidance, recommendations, and the like can also be determined using processor 191 and exported to output device 193 as well, as described further herein.

Sensor 104 is adapted to be at least partially inserted into a tissue of interest, such as within the dermal layer of the skin or in subcutaneous tissue. In some embodiments, the sensor can comprise a proximal portion configured to be positioned above a user's skin and a distal portion configured to be transcutaneously positioned through the user's skin and in contact with a bodily fluid. In some embodiments, the distal portion is configured to detect an analyte in the bodily fluid. In some embodiments, the proximal portion can be electrically coupled with processing electronics. In some embodiments, the processing electronics are disposed in the electronics housing of the sensor control device. Sensor 104 can comprise a sensor of sufficient length for insertion to a desired depth in a given tissue. The sensor can comprise a sensing region or sensing area that is active for sensing lactate, and can comprise a lactate-responsive enzyme, according to one or more embodiments. The sensing region or sensing area can include a polymeric material to which the lactate-responsive enzyme is covalently bonded, according to some embodiments. In some embodiments of the present disclosure, lactate can be monitored in any biological fluid of interest such as dermal fluid, plasma, blood, lymph, synovial fluid, cerebrospinal fluid, saliva, bronchoalveolar lavage, amniotic fluid, or the like. In some embodiments, lactate-responsive sensors of the present disclosure can be adapted for interrogating dermal fluid or interstitial fluid.

An introducer can be present transiently to promote introduction of sensor 104 into a tissue. In illustrative embodiments, the introducer can comprise a needle. It is to be recognized that other types of introducers, such as sheaths or blades, can be present in alternative embodiments. More specifically, the needle or similar introducer can transiently reside in proximity to sensor 104 prior to insertion and then be withdrawn afterward. While present, the needle or other introducer can facilitate insertion of sensor 104 into a tissue by opening an access pathway for sensor 104 to follow. For example, the needle can facilitate penetration of the epidermis as an access pathway to the dermis to allow implantation of sensor 104 to take place, according to some embodiments. After opening the access pathway, the needle or other introducer can be withdrawn so that it does not represent a sharps hazard. In some embodiments, the needle can be solid or hollow, beveled or non-beveled, and/or circular or non-circular in cross-section. In some embodiments, the needle can be comparable in cross-sectional diameter and/or tip design to an acupuncture needle, which can have a cross-sectional diameter of about 250 microns, for example. It is to be recognized, however, that suitable needles can have a larger or smaller cross-sectional diameter if needed for particular applications.

In some embodiments, a tip of the needle can be angled over the terminus of sensor 104, such that the needle penetrates a tissue first and opens an access pathway for sensor 104. In some embodiments, sensor 104 can reside within a lumen or groove of the needle, with the needle similarly opening an access pathway for sensor 104. In either case, the needle is subsequently withdrawn after facilitating insertion.

Sensor 104 can employ a two-electrode or a three-electrode detection motif, according to some embodiments of the present disclosure. Three-electrode motifs can comprise a working electrode, a counter electrode, and a reference electrode. Two-electrode motifs can comprise a working electrode and a second electrode, in which the second electrode functions as both a counter electrode and a reference electrode (i.e., a counter/reference electrode). In both two-electrode and three-electrode detection motifs, the sensing region or sensing area of sensor 104 can be in contact with the working electrode. In some embodiments, the electrodes can be at least partially stacked upon one another, as described in further detail hereinafter. In some embodiments, the various electrodes can be spaced apart from one another upon the insertion tail of sensor 104.

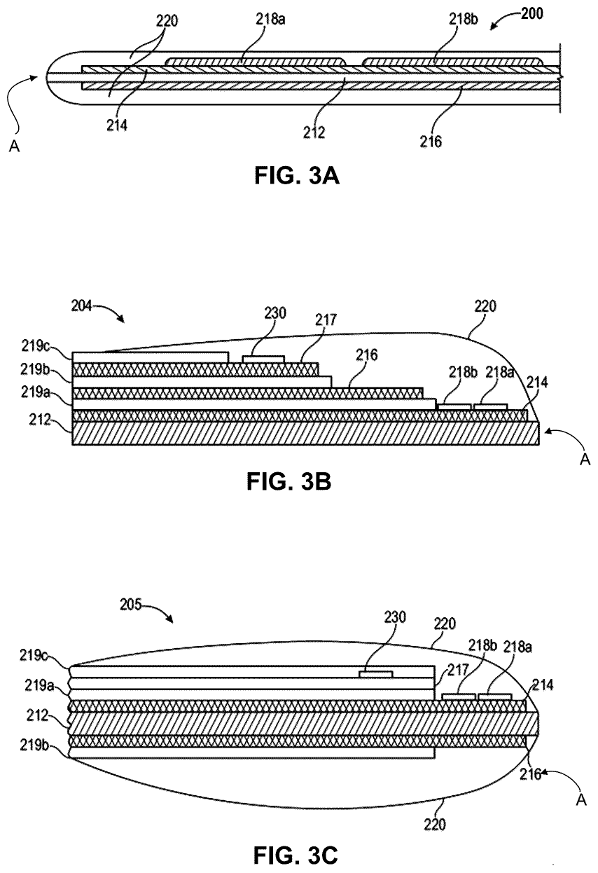

In some embodiments, the sensor can comprise sensing areas of the same type (e.g., two lactate-responsive sensing areas) upon a single working electrode or upon two or more separate working electrodes. Single working electrode sensor configurations can employ two-electrode or three-electrode detection motifs, according to some embodiments of the present disclosure and as described further herein. FIG. 3A shows a cross-sectional diagram of an illustrative two-electrode analyte sensor configuration having a single working electrode, which is compatible for use in some embodiments of the disclosure herein. As shown, analyte sensor 200 comprises substrate 212 disposed between working electrode 214 and counter/reference electrode 216. Alternately, working electrode 214 and counter/reference electrode 216 can be located upon the same side of substrate 212 with a dielectric material interposed in between (configuration not shown). In some embodiments, the sensor includes sensing areas 218a and 218b (e.g., both lactate-responsive sensing areas) that are laterally spaced apart from one another upon the surface of working electrode 214. In some embodiments, sensing areas 218a and 218b can be continuously disposed or discontinuously disposed on the working electrode for detection of each analyte. Analyte sensor 200 can be operable for assaying lactate by any of coulometric, amperometric, voltammetric, or potentiometric electrochemical detection techniques.

When a single working electrode is present in an analyte sensor, three-electrode sensor configurations can comprise a working electrode, a counter electrode, and a reference electrode. Related two-electrode sensor configurations can comprise a working electrode and a second electrode, wherein the second electrode can function as both a counter electrode and a reference electrode (i.e., a counter/reference electrode). In both two-electrode and three-electrode sensor configurations, both the first analyte-responsive sensing area and the second analyte-responsive sensing area can be disposed upon the single working electrode. In any of the sensor configurations disclosed herein, the various electrodes can be at least partially stacked (layered) upon one another and/or laterally spaced apart from one another upon the sensor. Suitable sensor configurations can be substantially flat in shape or substantially cylindrical in shape, with the first analyte-responsive sensing area and the second analyte-responsive sensing area being laterally spaced apart upon the working electrode. In all of the sensor configurations disclosed herein, the various electrodes can be electrically isolated from one another by a dielectric material or similar insulator.

In some embodiments, the sensor can comprise two or more working electrodes and at least one additional electrode (i.e., at least one additional electrode, at least two additional electrodes, etc.). When one additional electrode is present, the one additional electrode can function as a counter/reference electrode for each of the working electrodes. When two additional electrodes are present, one of the additional electrodes can function as a counter electrode for each of the working electrodes and the other additional electrode can function as a reference electrode for each of the working electrodes. FIGS. 3B-3C show diagrams of illustrative three-electrode analyte sensor configurations, which are also compatible for use in some embodiments of the disclosure herein. Three-electrode analyte sensor configurations can be similar to that shown for analyte sensor 200 in FIG. 3A, except for the inclusion of additional electrode 217 in analyte sensors 204 and 205 (FIGS. 3B-3C). With additional electrode 217, counter/reference electrode 216 may then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, as depicted in FIG. 3B, dielectric layers 219a, 219b and 219c separate electrodes 214, 216, and 217 from one another and provide electrical isolation. Alternately, at least one of electrodes 214, 216, and 217 can be located upon opposite faces of substrate 212, as shown in FIG. 3C. Thus, in some embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to the locations depicted in FIGS. 3B and 3C. As with sensor 200 shown in FIG. 3A, sensing areas 218a, 218b in analyte sensors 204, 205 can be continuously disposed or discontinuously disposed on the working electrode for detection of lactate. Additionally, analyte sensors 204, 205 can be operable for assaying lactate by any of coulometric, amperometric, voltammetric, or potentiometric electrochemical detection techniques.

Like analyte sensor 200, membrane 220 can also overcoat sensing areas 218a and 218b, as well as other sensor components, in analyte sensors 204, 205, thereby serving as a mass transport limiting membrane. Additional electrode 217 can be overcoated with membrane 220 in some embodiments. Although FIGS. 3B-3C have depicted all of electrodes 214, 216, and 217 as being overcoated with membrane 220, it is to be recognized that only working electrode 214 may be overcoated in some embodiments. Moreover, the thickness of membrane 220 at each of electrodes 214, 216 and 217 may be the same or different. As in two-electrode analyte sensor configurations (FIG. 3A), one or both faces of analyte sensors 204, 205 can be overcoated with membrane 220 in the sensor configurations of FIGS. 3B-3C, or the entirety of analyte sensors 204, 205 can be overcoated.

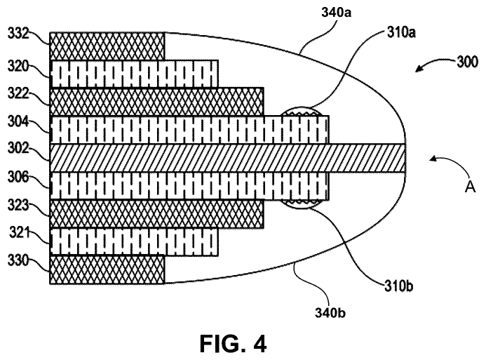

FIG. 4 shows a cross-sectional diagram of an illustrative analyte sensor configuration having two working electrodes, a reference electrode and a counter electrode, which is compatible for use in some embodiments of the disclosure herein. As shown in FIG. 4, analyte sensor 300 includes working electrodes 304 and 306 disposed upon opposite faces of substrate 302. Sensing area 310a is disposed upon the surface of working electrode 304, and sensing area 310b is disposed upon the surface of working electrode 306. Analyte sensor 300 can comprise additional sensing areas, which can be laterally spaced apart from one another upon the surface(s) of working electrode(s) 304 and/or 306. Sensing areas 310a and 310b can both be lactate-responsive sensing areas, according to some embodiments of the present disclosure. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. The layers present in the sensor 300 of FIG. 4 can comprise outer dielectric layer 332, counter electrode 320, dielectric layer 322, working electrode 304, substrate 302, working electrode 306, dielectric layer 323, reference electrode 321 and outer dielectric layer 330, in that order. The layers can be (partially) stacked on one another, with a part of each layer proximate an edge of said layer not covered by an adjacent layer. For example, substrate 302 can extend beyond (not be covered by) respective ends of working electrodes 304, 306. Working electrodes 304, 306 can extend beyond (not be covered by) the ends of dielectric layers 322, 333. Portions of the working electrodes 304, 306 that extend beyond the ends of the dielectric layers 322, 333 can provide a space for the sensing areas 310a, 310b, respectively. In any of the embodiments described herein, substrate 302 can extend to a distal tip of the sensor, so as to separate the layers on either side of the substrate 302. Membrane 340 has first membrane portion 340a and second membrane portion 340b, which separately overcoat at least sensing areas 310a and 310b, respectively, according to various embodiments, with other components of analyte sensor 300 or the entirety of analyte sensor 300 optionally being overcoated with first membrane portion 340a and/or second membrane portion 340b as well. Again, membrane 340 can be continuous but vary compositionally within first membrane portion 340a and second membrane portion 340b (i.e., upon sensing areas 310a and 310b) in order to afford different permeability values for differentially regulating the analyte flux at each location. For example, different membrane formulations can be sprayed and/or printed onto the opposing faces of analyte sensor 300. Dip coating techniques can also be appropriate, particularly for depositing at least a portion of a bilayer membrane upon one of sensing areas 310a and 310b. Accordingly, one of first membrane portion 340a and second membrane portion 340b can comprise a bilayer membrane and the other of first membrane portion 340a and second membrane portion 340b can comprise a single membrane polymer, according to some embodiments of the present disclosure. In some embodiments, analyte sensor 300 can be operable for assaying lactate by any of coulometric, amperometric, voltammetric, or potentiometric electrochemical detection techniques. The above description of the membrane applies equally to any of the sensor embodiments described herein.

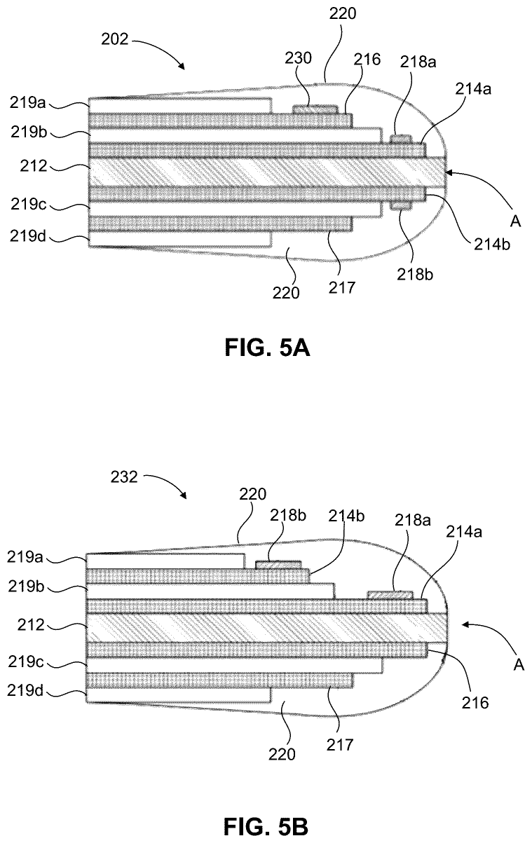

FIG. 5A shows a diagram of an illustrative four-electrode analyte sensor configuration, which is compatible for use in the disclosure herein. As shown, sensor 202 comprises substrate 212 disposed between working electrodes 214a and 214b. Alternately, working electrodes 214a and 214b can be located on the same side of substrate 212 with a dielectric material interposed in between (configuration not shown). Analyte-specific responsive sensing areas 218a and/or 218b (e.g., dual lactate-responsive sensing areas) can be disposed as at least one layer upon at least a portion of working electrodes 214a and/or 214b. The analyte-responsive sensing area(s) can be continuously disposed or discontinuously disposed on the working electrode(s) for detection of the analyte, as discussed further herein. A reference electrode can be disposed upon either working electrodes 214a or 214b, with a separating layer of dielectric material in between. A counter electrode can be disposed on the other side of working electrodes 214a or 214b, with a separating layer of dielectric material in between. For example, as depicted in FIG. 5A, dielectric layers 219b and 219c separate electrodes 214a, 214b, 216, and 217 from one another and provide electrical isolation. Outer dielectric layers 219a and 219d are positioned on reference electrode 216 and counter electrode 217. In other embodiments, at least one of electrodes 214a, 214b, 216, and 217 can be located upon opposite faces of substrate 212 (configuration not shown).

In some embodiments, electrode 214a (working electrode) and electrode 216 (counter electrode) can be located upon opposite faces of substrate 212 as electrode 217 (reference electrode), with working electrode 214b being located on the opposite face of the substrate as depicted in FIG. 5A. Reference material layer 230 (e.g., Ag/AgCl) can be present upon reference electrode 216, with the location of reference material layer 230 not being limited to that depicted in FIG. 5A. Additionally, analyte sensor 202 can be operable for assaying the analyte by any of coulometric, amperometric, voltammetric, or potentiometric electrochemical detection techniques. Although FIG. 5A has depicted all of electrodes 214a, 214b, 216, and 217 as being overcoated with membrane 220, it is to be recognized that only working electrodes 214a and 214b can be overcoated in some embodiments. Moreover, the thickness of membrane 220 at each of electrodes 214a, 214b, 216, and 217 can be the same or different, in any of the embodiments described herein. As in two-electrode analyte sensor configurations, one or both faces of analyte sensor 202 can be overcoated with membrane 220 in the sensor configurations of FIG. 5A, or the entirety of analyte sensors 202 can be overcoated. Accordingly, the multiple-electrode sensor configuration shown in FIG. 5A should be understood as being non-limiting of the embodiments disclosed herein, with alternative electrode and/or layer configurations remaining within the scope of the present disclosure.

In some embodiments (including any of the embodiments of FIGS. 3-6), the substrate is formed from any suitable inert material. In some embodiments, the substrate is biocompatible. Examples of a suitable substrate include titanium, a carbon-based substrate (e.g., cellulose, polylactic acid) and a plastic substrate (e.g., polyethylene terephthalate, polyethylene, polypropylene, polymethylmethacrylate, polysulfone, polydimethylsiloxane, polyvinyl chloride, etc.). The substrate can be disposed between the working electrode and a counter and/or reference electrode.

In some embodiments (including any of the embodiments of FIGS. 3-6), the dielectric layer can comprise a suitable dielectric material that can form a solid. In an example, the insulation layer can be formed from porcelain (ceramic), mica, glass, barium strontium titanate, a plastic (e.g., polystyrene, polytetrafluoroethylene, polyethylene terephthalate, polyethylene, polypropylene, polymethylmethacrylate, polysulfone, polydimethylsiloxane, polyvinyl chloride), or a metal oxide (e.g., silica, alumina, titania, zirconia, tantalum oxide, etc.).

In some embodiments (including any of the embodiments of FIGS. 3-6), membrane 220 optionally overcoats at least analyte-responsive sensing areas 218a and 218b and overcoats some or all of working electrodes 214a and/or 214b and/or reference electrode 216 and/or counter electrode 217, or the entirety of analyte sensor 202 according to some embodiments. One or both faces of analyte sensor 202 can be overcoated with membrane 220. Membrane 220 can comprise one or more polymeric membrane materials having capabilities of limiting analyte flux to sensing areas 218a, 218b (i.e., membrane 220 is a mass transport limiting membrane having some permeability for the analyte(s) being measured). The composition and thickness of membrane 220 can vary to promote a desired analyte flux to analyte-responsive sensing areas 218a, 218b, thereby providing a desired signal intensity and stability.

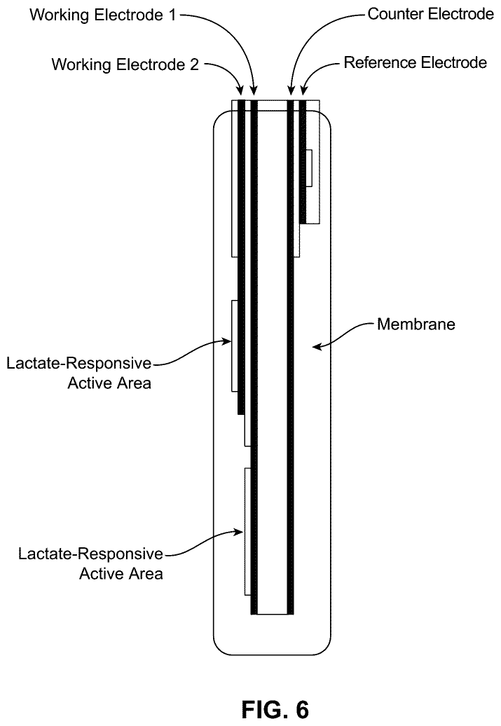

FIG. 5B and FIG. 6 show diagrams of an illustrative four-electrode analyte sensor configuration, which is compatible for use in the disclosure herein. As shown, analyte sensor 232 comprises substrate 212 disposed between working electrode 214a and counter electrode 216. Working electrodes 214a and 214b are located on the same side of substrate 212 with a dielectric material 219b interposed in between working electrodes 214a and 214b. Counter electrode 216 and reference electrode 217 are located on the opposite side of substrate 212 with a dielectric material 219c interposed in between counter electrode 216 and reference electrode 217. Analyte-specific responsive sensing area 218a (e.g., lactate-responsive sensing area) can be disposed as at least one layer upon at least a portion of working electrode 214a. Analyte-specific responsive sensing area 218b (e.g., lactate-responsive sensing area) can be disposed as at least one layer upon at least a portion of working electrode 214b. Sensing area 218a (e.g., lactate-responsive sensing area) can be located closer to end A than analyte-specific responsive sensing area 218b (e.g., lactate-responsive sensing area). The analyte-responsive sensing area(s) 218a and 218b can be continuously disposed or discontinuously disposed on the working electrode(s) for detection of the analyte, as discussed further herein. In some embodiments, the analyte-responsive sensing areas 218a, 218b can both be a single spot. Dispensing both sensing areas (analyte-responsive sensing areas 218a, 218b) on one side of substrate 212 without needing to flip the substrate 212 can simplify the manufacturing process and improves efficiency. As depicted in FIG. 5B, dielectric layers 219b and 219c separate electrodes 214a, 214b, 216, and 217 from one another and provide electrical isolation. Outer dielectric layers 219a and 219d are positioned on working electrode 214b and counter electrode 217. Reference material layer 230 (e.g., Ag/AgCl) (not shown) can be present upon reference electrode 216, or another suitable location on the sensor. The layers present in the sensor 232 of FIG. 5B may comprise or consist of outer dielectric layer 219a, working electrode 214b, dielectric layer 219b, working electrode 214a, substrate 212, reference electrode 216, dielectric layer 219c, counter electrode 217 and outer dielectric layer 219d, in that order. The layers can be (partially) stacked on one another, with a part of each layer proximate an end of said layer not covered by an adjacent layer. Additionally, analyte sensors 202, 232 can be operable for assaying the analyte by any of coulometric, amperometric, voltammetric, or potentiometric electrochemical detection techniques.

In some embodiments, sensor 232 can contain a membrane 220. Membrane 220 can cover both sensing area 218a (e.g., a lactate-responsive sensing area) and sensing area 218b (e.g., a lactate-responsive sensing area). Membrane 220 can also cover counter electrode 216 and reference electrode 217 on the opposite side of substrate 212. Although FIG. 5B has depicted all of electrodes 214a, 214b, 216, and 217 as being overcoated with membrane 220, it is to be recognized that only working electrodes 214a and 214b can be overcoated in some embodiments. In some embodiments, membrane 220 can be dip coated onto sensing areas 218a, 218b (e.g., dual lactate-responsive sensing areas) and the electrodes 214a, 214b, 216, and 217. As in two-electrode analyte sensor configurations, one or both faces of analyte sensor 232 can be overcoated with membrane 220 in the sensor configurations of FIG. 5B, or the entirety of analyte sensor 232 can be overcoated. Accordingly, the multiple-electrode sensor configuration shown in FIGS. 5A and 5B should be understood as being non-limiting of the embodiments disclosed herein, with alternative electrode and/or layer configurations remaining within the scope of the present disclosure.

In some embodiments, the membrane 220 can comprise at least a crosslinked polyvinylpyridine homopolymer or copolymer. The membrane 220 can be single-component or multi-component. Multi-component membrane embodiments can comprise a bilayer or homogeneous admixture of the crosslinked polyvinylpyridine and another polymer, according to some embodiments of the present disclosure. Suitable polyvinylpyridine copolymers for inclusion in the membrane 220 can comprise up to about 25% comonomers (based on the total amount of monomers in the copolymer), such as from about 0.1% to about 5% comonomers, or about 5% to about 15% comonomers, or about 15% to about 25% comonomers, or about 1% to about 10% co-monomers. Suitable comonomers are not particularly limited, provided that the mass transport limiting membrane affords sufficient lactate permeability to provide an analyte sensitivity of about 1 nA/mM or greater when exposed to lactate. The polyvinylpyridine copolymer can be distinct from a polyvinylpyridine-co-styrene copolymer, according to some embodiments. Crosslinking of these membrane polymers in an analyte sensor can take place through functionalization with a bis-epoxide, such as polyethylene glycol diglycidyl ether (PEGDGE) or glycerol triglycidyl ether. In some embodiments, membrane 220 can comprise polyvinylpyridine and a crosslinker, such as polyethylene glycol diglycidyl ether (PEGDGE), e.g., PEGDGE 400.

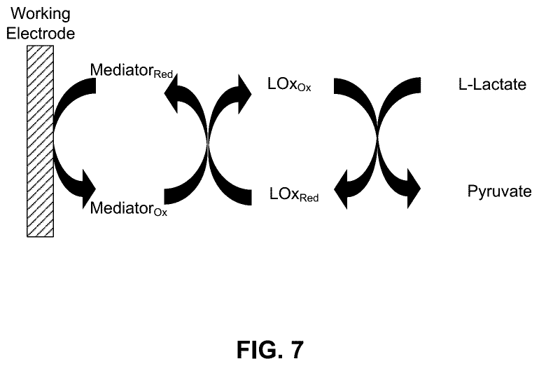

In some embodiments, sensing area 218a and sensing area 218b can each comprise a lactate-responsive enzyme. More particularly, the lactate-responsive enzyme can comprise lactate dehydrogenase or lactate oxidase, according to some embodiments of the present disclosure. FIG. 7 shows a diagram of an enzyme system that can be used for detecting lactate according to the disclosure herein. In some embodiments, an electron transfer mediator in each sensing area 218a, 218b can facilitate conveyance of electrons from lactate to the working electrodes 214a, 214b during a redox reaction as shown in FIG. 7. The electrons transferred during this reaction provide the basis for lactate detection in the working electrode. Changes in the signal intensity (e.g., current) at working electrodes 214a, 214b can be proportional to the lactate concentration and/or the activity of the lactate-responsive enzyme.

In some embodiments, sensing areas 218a, 218b can each further comprise a stabilizer for lactate dehydrogenase or lactate oxidase, such as catalase or albumin. According to some embodiments, the lactate-responsive enzyme, such as lactate dehydrogenase or lactate oxidase, can be covalently bonded to a polymer in sensing areas 218a, 218b. Covalent bonding immobilizes the lactate-responsive enzyme in sensing areas 218a, 218b.

In some embodiments, lactate oxidase can be present in the sensing area in an amount ranging from about 0.05 μg to about 5 μg, or from about 0.1 μg to about 4 μg, or from about 0.2 μg to about 3 μg, or from about 0.5 μg to about 2 μg. In terms of weight percentage of the sensing area, the lactate oxidase can be present in an amount ranging from about 10% to about 90% by weight of the sensing area, or from about 25% to about 75% by weight of the sensing area, or from about 30% to about 60% by weight of the sensing area.

According to some embodiments, the albumin within the sensing area can comprise human serum albumin. In some embodiments, non-human albumin such as bovine serum albumin, can be used.

The albumin can be incorporated within the sensing area in an amount sufficient to stabilize the lactate-responsive enzyme, particularly lactate oxidase. In more specific embodiments, the albumin can be present in the sensing area in an amount ranging from about 0.05 μg to about 5 μg, or from about 0.1 μg to about 2 μg, or from about 0.2 μg to about 1.5 μg, or from about 0.3 μg to about 0.8 μg. In terms of weight percentage of the sensing area, the albumin can be present in an amount ranging from about 25% to about 75% by weight of the sensing area, or from about 30% to about 60% by weight of the sensing area. In certain embodiments, the weight ratio of lactate oxidase to albumin can range from about 10:1 to about 1:10 (w/w), or from about or from about 5:1 to about 1:5, or from about 5:1 to about 1:1, or from about 2:1 to about 1:1, or from about 1:1 to about 1:5, or from about 1:1 to about 1:2. In some embodiments, the weight ratio of lactate oxidase to albumin can be about 2:1. In some embodiments, the weight ratio of lactate oxidase to albumin can be about 1:1.

In some embodiments, sensing areas 218a, 218b can each comprise a redox mediator that can comprise a polymer and an electron transfer agent. In some embodiments, the electron transfer agent can be a low-potential osmium complex electron transfer mediator. In some embodiments, the polymer is covalently bonded to both the lactate-responsive enzyme, such as lactate dehydrogenase or lactate oxidase, and a low-potential osmium complex electron transfer mediator, as disclosed in, for example, U.S. Pat. Nos. 6,134,461, 6,605,200, 6,736,957, 7,501,053, and 7,754,093, the disclosures of each of which are incorporated herein by reference in their entirety. Other suitable examples of electron transfer mediators and polymer-bound electron transfer mediators can include those described in U.S. Pat. Nos. 8,444,834, 8,268,143, and 6,605,201, the disclosures of which are also incorporated herein by reference in their entirety.

The electron transfer mediator can facilitate conveyance of electrons from lactate to working electrodes 214a, 214b during a redox reaction. Changes in the signal intensity (e.g., current) at working electrodes 214a, 214b can be proportional to the lactate concentration and/or the activity of the lactate-responsive enzyme. In some embodiments, a calibration factor can be applied (e.g., by a processor) to determine the lactate concentration from the signal intensity. Suitable electron transfer mediators include electroreducible and electrooxidizable ions, complexes or molecules having redox potentials that are a few hundred millivolts above or below the redox potential of the standard calomel electrode (SCE). Other suitable electron transfer mediators can comprise metal compounds or complexes of ruthenium, iron (e.g., polyvinylferrocene), or cobalt, for example. Suitable ligands for the metal complexes can include, for example, bidentate or higher denticity ligands such as, for example, a bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate, or higher denticity ligands can be present in the metal complex to achieve a full coordination sphere.

The polymer in the redox mediator can be any suitable polymer that allows the transfer of electrons between the electron transfer agent and the working electrode. For example, the polymer can be, poly(4-vinylpyridine), poly(l-vinylimidazole), poly(thiophene), poly(aniline), poly(pyrrole), poly(acetylene), poly(acrylic acid), styrene/maleic anhydride copolymer, methylvinylether/maleic anhydride copolymer, poly(vinylbenzylchloride), poly(allylamine), poly(lysine), poly(acrylamide-co-1-vinyl imidazole), poly(4-vinylpyridine) quaternized with carboxypentyl groups, and poly(sodium 4-styrene sulfonate). These polymers can be considered precursor polymers in that the polymers are further modified to immobilize (e.g., attach) the electron transfer complex. In some embodiments, the polymer can comprise a poly(4-vinylpyridine), poly(l-vinylimidazole), poly(thiophene), poly(aniline), poly(pyrrole), or poly(acetylene) backbone. In other embodiments, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. For example, a suitable polymer includes partially or fully quaternized poly(4-vinylpyridine) and poly(1-vinylimidazole) in which quaternized pyridine and imidazole groups, respectively, can be used to form spacers by reaction with (e.g., complexation with) an electron transfer agent.

Suitable polymers for inclusion in sensing areas 218a, 218b include, but are not limited to, polyvinylpyridines (e.g., poly(4-vinylpyridine)), polyimidazoles (e.g., poly(1-vinylimidazole), or any copolymer thereof. Illustrative copolymers that can be suitable include, for example, copolymers containing monomer units such as styrene, acrylamide, methacrylamide, or acrylonitrile.

Covalent bonding of the lactate-responsive enzyme to a polymer or other matrix (e.g., sol-gel) in sensing areas 218a, 218b can take place via a crosslinker introduced with a suitable crosslinking agent. Suitable crosslinking agents for reaction with free amino groups in the enzyme (e.g., with the free amine in lysine) can include crosslinking agents such as, for example, polyethylene glycol diglycidylether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme can include, for example, carbodiimides.

In some embodiments, the redox mediator can comprise an osmium complex bonded to a polymer or copolymer of poly(1-vinyl imidazole) or poly(4-vinylpyridine). The poly(4-vinylpyridine)-based polymer is a prepolymer that has been modified, as shown in the following structure, to attach an osmium complex (e.g., a poly(biimidizyl) osmium complex).

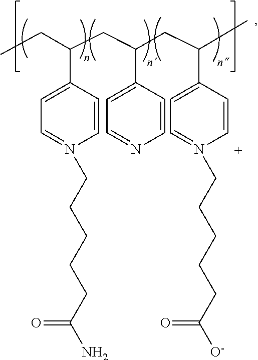

wherein n can be 2, n′ can be 17, and n′ can be 1. Other reactive groups and/or spacer groups can be used.

In some embodiments, the electron redox mediator can comprise an osmium-containing poly(4-vinylpyridine)-based polymer, as shown below.

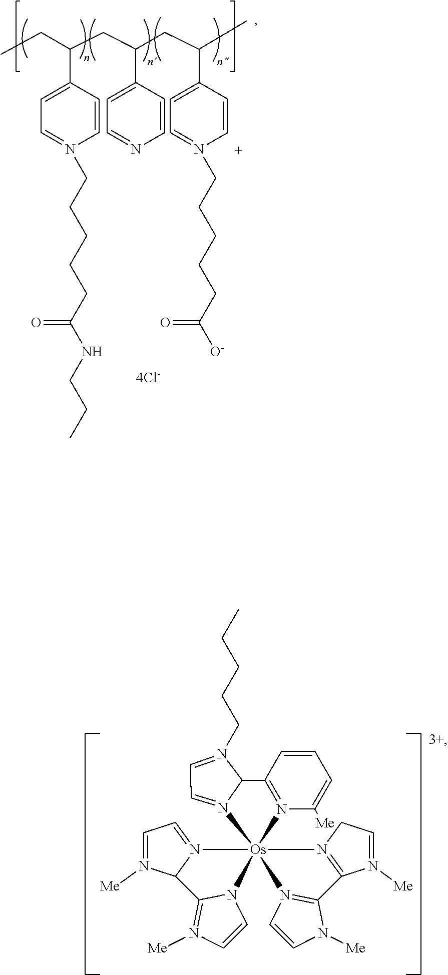

wherein n is 2, n′ is 17, and n″ is 1.

Although the lactate-responsive enzyme and/or the electron transfer mediator can be covalently bonded to a polymer or other suitable matrix in sensing area 218a, other association means can be suitable as well. In some embodiments, the lactate-responsive enzyme and/or the electron transfer mediator can be ionically or coordinatively associated with the polymer or other matrix. For example, a charged polymer can be ionically associated with an oppositely charged lactate-responsive enzyme or electron transfer mediator. In still other embodiments, the lactate-responsive enzyme and/or the electron transfer mediator can be physically entrained within the polymer or other matrix of sensing area 218a.

In some embodiments, the first sensing area 218a can comprise a lactate-responsive enzyme, such as lactate oxidase, and the second sensing area 218b can also comprise a lactate-responsive enzyme, such as lactate oxidase, in addition to the suitable electron transfer agents and polymers discussed in more detail above. According to some embodiments, the sensor can be suitable for detecting lactate and can comprise a first working electrode 214a having a first sensing area 218a disposed thereon and a second working electrode having a second sensing area 218b disposed thereon, and a mass transport limiting membrane overcoating the first and second sensing areas upon the working electrode, in which the first sensing area 218a comprises a polymer, an albumin, and a lactate-responsive enzyme (e.g., lactate oxidase) covalently bonded to the polymer and the second sensing area 218b comprises a lactate-responsive enzyme (e.g., lactate oxidase) covalently bonded to a polymer. In some embodiments, the first and second electron transfer agents can be the same. In some embodiments, the first and second electron transfer agents can be different from one another. In some embodiments, the mass transport limiting membrane can comprise at least a crosslinked polyvinylpyridine homopolymer or copolymer.

In some embodiments, the sensor discussed herein can be configured to be partially inserted into a user's skin. The working electrodes are disposed upon the sensor and can be inserted in the tissue to facilitate lactate and glucose analyses therein. Suitable tissues are not considered to be particularly limited and specific examples are addressed in more detail above. Similarly, considerations for deploying a sensor at a particular position or depth within a tissue are addressed above.

Detection methods for assaying lactate employing the sensor disclosed herein can comprise: exposing an analyte sensor to a fluid comprising lactate; applying a potential to the first working electrode and the second working electrode, obtaining a first signal at or above an oxidation-reduction potential of the first lactate-responsive sensing area, in which the first signal is proportional to a concentration of lactate in the fluid, obtaining a second signal at or above an oxidation-reduction potential of the second lactate-responsive sensing area, in which the second signal is proportional to a concentration of lactate in the fluid, and correlating the first signal to the concentration of lactate in the fluid and the second signal to the concentration of lactate in the fluid. In some embodiments, the first signal and the second signal are obtained via a first channel and a second channel to allow independent production of signals from the first and second lactate-responsive sensing areas. In some embodiments, the first signal and the second signal are obtained simultaneously via a first channel and a second channel to allow independent production of signals from the first and second lactate-responsive sensing areas.

In some embodiments, the signals associated with each sensing area can be correlated to a corresponding concentration of lactate by consulting a lookup table or calibration curve for lactate. A lookup table for lactate can be populated by assaying multiple samples having known analyte concentrations and recording the sensor response at each concentration for lactate. Similarly, a calibration curve for lactate can be determined by plotting the analyte sensor response for lactate as a function of the concentration and determining a suitable calibration function over the calibration range (e.g., by regression, particularly linear regression).

A processor can determine which sensor response value in a lookup table is closest to that measured for a sample having an unknown analyte concentration and then determine whether to report the analyte concentration based on the algorithm discussed below. In some embodiments, if the sensor response value for a sample having an unknown analyte concentration is between the recorded values in the lookup table, the processor can interpolate between two lookup table values to estimate the analyte concentration. Interpolation can assume a linear concentration variation between the two values reported in the lookup table. Interpolation can be employed when the sensor response differs a sufficient amount from a given value in the lookup table, such as variation of about 10% or greater.

Likewise, according to some embodiments, a processor can input the sensor response value for a sample having an unknown analyte concentration into a corresponding calibration function. The processor can then report the analyte concentration accordingly.

In some embodiments, the sensors described herein can also be configured to analyze for other analytes as well. Other analytes commonly subject to physiological dysregulation that can similarly be desirable to monitor include, but are not limited to, glucose, oxygen, pH, A1c, ketones, drug levels, and the like. In some embodiments, the sensor includes dual glucose-responsive sensing areas instead of lactate-responsive sensing areas to independently produce signals indicative of glucose concentrations. The description of the embodiments herein applies equally to sensors having dual glucose-responsive sensing areas, unless obviously incompatible.

It is to be appreciated that the sensing system and sensor disclosed herein can comprise additional features and/or functionality that are not necessarily described herein in the interest of brevity. Thus, the foregoing description of the sensing system and sensor should be considered illustrative and non-limiting in nature.

### Methods

Also disclosed herein are methods for monitoring lactate levels in an individual and methods for determining variance between simultaneous lactate concentration measurements in a sensor having two difference channels. Without wishing to be bound by a particular theory, signal abnormality and variance between two sensors positioned on the same substrate may be caused by factors including, but not limited to, varying degrees of impact of early signal rise and late signal rise on the two channels, physical issues related to sensor/channel integrity, and partial insertion of the upper electrode into the interstitial fluid (e.g., the second working electrode). The methods disclosed herein can detect variance between the channels so that a sensing system can report accurate lactate concentration data from at least one of the channels to a user.

In some embodiments, the method for monitoring lactate levels in an individual comprises exposing an analyte sensor of a sensing system to a fluid. In some embodiments, the sensor can be any sensor as disclosed herein. In some embodiments, the analyte sensor can be one of the sensors described in relation to FIGS. 5B and 6. In some embodiments, the method further comprises: applying a potential to a first working electrode of the sensor; obtaining a first signal at or above an oxidation-reduction potential of a first lactate-responsive sensing area disposed on the first working electrode, the signal being proportional to a concentration of lactate in the fluid; applying a potential to a second working electrode of the sensor; obtaining a second signal at or above an oxidation-reduction potential of a second lactate-responsive sensing area disposed on the second working electrode, the second signal being proportional to a concentration of lactate in the fluid; and correlating the first and second signals to concentrations of lactate in the fluid.

In some embodiments, each sensing area has an oxidation-reduction potential, and the oxidation-reduction potential of the first lactate-responsive sensing area is sufficiently separated from the oxidation-reduction potential of the second lactate-responsive sensing area to allow independent production of signals from the first and second the lactate-responsive sensing areas.

In some embodiments, the method for determining variance in lactate concentration measurements in a sensing system comprising a lactate-responsive sensor, can comprise continuously measuring signals indicative of lactate concentrations in a biological fluid in vivo with the lactate-responsive sensor. As discussed above, in some embodiments, the sensor can be any sensor as disclosed herein. In some embodiments, the lactate-responsive sensor includes a first lactate-responsive sensing area disposed on a first working electrode and a second lactate-responsive sensing area disposed on a second working electrode to simultaneously and independently obtain first and second signals indicative of lactate concentrations measured at the first and second working electrodes.

In some embodiments, the sensor is configured to take a simultaneous measurement of the first and second signals indicative of lactate concentrations measured by the first and second working electrodes, respectively, about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours. In some embodiments, the sensor is configured to take a measurement about every minute. In some embodiments, the method can further comprise communicating the first and second signals indicative of lactate concentrations measured at the first and second working electrodes to the processor.

In some embodiments, the processor can be configured to receive the first and second signals from the lactate-responsive sensor and determine lactate concentrations measured by the lactate-responsive sensor. In some embodiments, the processor is configured to correlate the first and second signals indicative of lactate concentrations measured simultaneously by the first and second working electrodes to corresponding first and second lactate concentrations.

In some embodiments, the processor is configured to compare the first and second lactate concentrations measured simultaneously by the first and second working electrodes and determine variance between the first and second lactate concentrations. In some embodiments, variance is determined within a detection window. In some embodiments, the detection window can be about 1 hour to about 5 hours. In some embodiments, the detection window can be about 1 hour about to about 4 hours, about 1 hour to about 3 hours, about 1 hour to about 2 hours. In some embodiments, the detection window can be about 2 hour about to about 5 hours, about 2 hour to about 4 hours, about 2 hour to about 3 hours. In some embodiments, the detection window can be about 1 hour, about 2 hours, about 3 hours, about 4 hours, or about 5 hours. In some embodiments, the detection window can be about 3 hours.

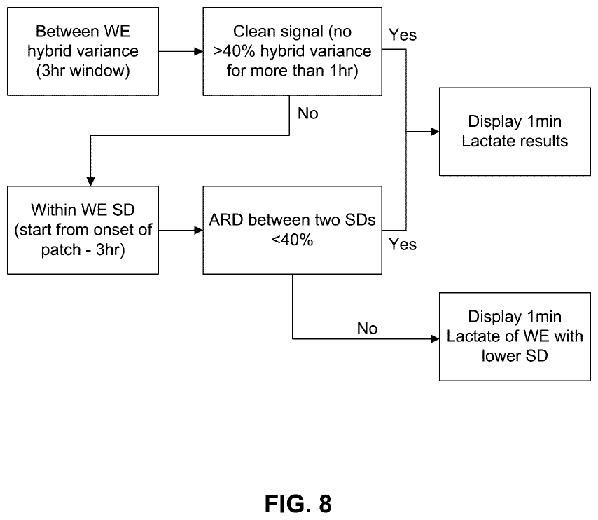

In some embodiments, the processor can be configured to determine variance between the first and second lactate concentrations by the algorithm shown in FIG. 8. That is, in some embodiments, the processor is configured to determine variance by first determining a hybrid variance based on the first and second lactate concentrations measured simultaneously by the first and second working electrodes. The processor may be configured to determine the mean of the first and second lactate concentrations, and select the formula (used to determine hybrid variance) based on the determined mean of the first and second lactate concentrations.

As used herein, hybrid variance refers to a variance between the first and second lactate concentration that is calculated in one of two ways depending on mean lactate concentration. If the mean of the first and second lactate concentrations is less than or equal to about 2.5 mM lactate (exemplary threshold), the hybrid variance is defined as a standard deviation of the first and second lactate concentrations multiplied by 40 (SD*40). Standard deviation is calculated as shown below.

$$σ = \sqrt{\frac{\sum {( {x}_{i} - μ )}^{2}}{N}}$$

σ: population standard deviation
N: size of the population
xᵢ: each value from the population
μ: population mean

In some embodiments, if the mean of the first and second lactate concentrations is greater than about 2 mM lactate to about 3 mM lactate, the hybrid variance can be defined as a standard deviation of the first and second lactate concentrations multiplied by 40 (SD*40). In some embodiments, if the mean of the first and second lactate concentrations is greater about 2 mM, about 2.05 mM, about 2.1 mM, about 2.15 mM, about 2.2 mM, about 2.25 mM, about 2.3 mM, about 2.35 mM, about 2.4 mM, about 2.45 mM, about 2.5 mM, about 2.55 mM, about 2.6 mM, about 2.65 mM, about 2.7 mM, about 2.75 mM, about 2.8 mM, about 2.85 mM, about 2.9 mM, about 2.95 mM, or about 3 mM lactate, the hybrid variance can be defined as a standard deviation of the first and second lactate concentrations multiplied by 40 (SD*40).

If the mean of the first and second lactate concentrations is greater than about 2.5 mM lactate (exemplary threshold), the hybrid variance is defined as a covariance (COV) of the first and second lactate concentrations multiplied by 100. Covariance is calculated as shown below.

$$COV = \frac{\vert ( {S}_{2} - {S}_{1} ) \vert}{{S}_{avg}} \times 100$$

S₁=first lactate concentration
S₂=second lactate concentration
S_{avg}=mean of first and second lactate concentrations

In some embodiments, if the mean of the first and second lactate concentrations is greater about 2 mM lactate to about 3 mM lactate, the hybrid variance can be defined as a covariance (COV) of the first and second lactate concentrations multiplied by 100, as shown above. In some embodiments, if the mean of the first and second lactate concentrations is greater about 2 mM, about 2.05 mM, about 2.1 mM, about 2.15 mM, about 2.2 mM, about 2.25 mM, about 2.3 mM, about 2.35 mM, about 2.4 mM, about 2.45 mM, about 2.5 mM, about 2.55 mM, about 2.6 mM, about 2.65 mM, about 2.7 mM, about 2.75 mM, about 2.8 mM, about 2.85 mM, about 2.9 mM, about 2.95 mM, or about 3 mM lactate, the hybrid variance can be defined as a covariance (COV) of the first and second lactate concentrations multiplied by 100, as shown above.

In some embodiments, as long as the hybrid variance is not greater than a given threshold, for example 40% (i.e. a covariance of 40% or a variance of 40), for more than a given detection window, for example about an hour, the processor is configured to transmit an average (or weighted average) of the first and second lactate concentrations to be displayed for a user. In some embodiments, the processor is configured to transmit an average of the first and second lactate concentrations to be displayed if the sensitivity of the signals indicative of lactate concentrations measured at the first and second working electrodes are about the same. In some embodiments, the processor is configured to transmit a weighted average of the first and second lactate concentrations to be displayed if the sensitivity of the signals indicative of lactate concentrations measured at the first and second working electrodes are different. In some embodiments, the weighted average can be weighted so that it comprises about 1% to about 99% of the first lactate concentration and about 99% to about 1% of the second lactate concentration for a total of 100%. In some embodiments, the first lactate concentration can be weighted so that it comprises about 5% to about 95%, about 10% to about 90%, about 15% to about 85%, about 20% to about 80%, about 25% to about 75%, about 30% to about 70%, about 35% to about 65%, about 40%, to about 60%, or about 45% to about 55% of the average. In some embodiments, the second lactate concentration can be weighted so that it comprises about 95% to about 5%, about 90% to about 10%, about 85% to about 15%, about 80% to about 20%, about 75% to about 25%, about 70% to about 30%, about 65% to about 35%, about 60%, to about 40%, or about 55% to about 45% of the average. In some embodiments, the weighted average can be weighted in favor of the first lactate concentration (e.g., 65% first lactate concentration and 35% second lactate concentration). In some embodiments, the weighted average can be weighed in favor of the second lactate concentration (e.g., 35% first lactate concentration and 65% second lactate concentration). In some embodiments, because the sensor continuously measures lactate (e.g., every minute), the processor continuously transmits (e.g., every minute) an average (or weighted average) of the first and second lactate concentrations derived from the first and second working electrodes to be displayed. In some embodiments, the output of the processor can be numerical and/or graphical. The notification to the wearer of the lactate-responsive sensor or other interested party can be auditory, tactile (haptic), or any combination thereof.

In some embodiments, if the hybrid variance is greater than the given threshold, for example 40%, for more than the given detection window, for example about an hour, a noisy patch has been detected (i.e., no longer a clean signal) and the processor is configured to calculate an absolute relative difference (ARD) in order to determine whether to display the first lactate concentration, the second lactate concentration, or an average or weighted average of the first and second lactate concentrations. In some embodiments, the ARD is defined as a difference between the standard deviation of a series of first lactate concentrations derived from the first working electrode over a given detection window (“SD₁”) and the standard deviation of a series of second lactate concentrations derived from the second working electrode over the same detection window (“SD₂”), all multiplied by 100. Mathematically, this formula is represented as follows: ARD=|SD₂−SD₁|×100. Standard deviation is calculated as described earlier herein.

In some embodiments, the ARD and standard deviation calculations are based on lactate concentrations from the onset of the noisy patch (e.g., from the initial point of when hybrid variance was determined to be greater than 40% for more than about an hour).

In some embodiments, the processor is configured to transmit an average (or weighted average) of the first and second lactate concentrations to be displayed if the ARD is less than or equal to an ARD threshold, for example about 40%. In some embodiments, if the ARD is greater than the ARD threshold, for example about 40%, the processor is configured to transmit the lactate concentration associated having the lower standard deviation.

In some embodiments, the detection window can be about 2 hours to about 5 hours starting from the onset of the noisy patch. In some embodiments, the detection window can be about 2 hours to about 4 hours, about 2 hours to about 4 hours, about 2 hours to about 3 hours, about 3 hours to about 5 hours, about 3 hours to about 4 hours, or about 4 hours to about 5 hours. In some embodiments, the detection window can be about 3 hours starting from the onset of the noisy patch. In some embodiments, the detection window can be a rolling detection window.

The present disclosure is further illustrated by the following embodiments.

(1) An analyte sensor comprising:
a substrate;
a first working electrode located on the substrate;
a second working electrode located on the substrate;
a first lactate-responsive sensing area disposed on a surface of the first working electrode;
a second lactate-responsive sensing area disposed on a surface of the second working electrode; and
a membrane that is permeable to lactate overcoating the first and second lactate-responsive sensing areas,
wherein the first and second lactate-responsive sensing areas are configured to independently produce first and second signals indicative of lactate concentrations measured at the first and second working electrodes, and
wherein the sensor is configured to be partially inserted into a user's skin.

(2) The analyte sensor of (1), wherein the first lactate-responsive sensing area comprises lactate oxidase.

(3) The analyte sensor of (1) or (2), wherein the first lactate-responsive sensing area comprises a first polymer and a first electron transfer agent.

(4) The analyte sensor of (3), wherein the first electron transfer agent is covalently bonded to the first polymer.

(5) The analyte sensor of (3) or (4), wherein the first electron transfer agent comprises an osmium complex.

(6) The analyte sensor of any one of (3-5), wherein the first polymer is a poly(4-vinylpyridine)-based polymer.

(7) The analyte sensor of any one of (3-6), wherein the first lactate-responsive sensing area further comprises a first crosslinker.

(8) The analyte sensor of (7), wherein the first crosslinker is polyethylene glycol diglycidyl ether (PEGDGE).

(9) The analyte sensor of any one of (1-8), wherein the second lactate-responsive sensing area comprises lactate oxidase.

(10) The analyte sensor of any one of (1-9), wherein the second lactate-responsive sensing area comprises a second polymer and a second electron transfer agent.

(11) The analyte sensor of (10), wherein the second electron transfer agent is covalently bonded to the second polymer.

(12) The analyte sensor of (10) or (11), wherein the second electron transfer agent comprises an osmium complex.

(13) The analyte sensor of any one of (10-12), wherein the second polymer is a poly(4-vinylpyridine)-based polymer.

(14) The analyte sensor of any one of (10-13), wherein the second lactate-responsive sensing area further comprises a second crosslinker.

(15) The analyte sensor of (14), wherein the second crosslinker is PEGDGE.

(16) The analyte sensor of any one of (1-7), wherein the sensor further comprises a reference electrode.

(17) The analyte sensor of (1-16), wherein the sensor further comprises a counter electrode.

(18) The analyte sensor of any one of (1-17), wherein the membrane comprises at least a crosslinked polyvinylpyridine homopolymer or copolymer.

(19) A method for monitoring lactate levels in an individual, comprising:
a) exposing an analyte sensor of a sensing system to a fluid; wherein the analyte sensor comprises:
   a substrate;
   a first working electrode located on the substrate;
   a second working electrode located on the substrate;
   a first lactate-responsive sensing area disposed on a surface of the first working electrode;
   a second lactate-responsive sensing area disposed on a surface of the second working electrode; and
   a membrane that is permeable to lactate overcoating the first and second lactate-responsive sensing areas,
   wherein the first and second lactate-responsive sensing areas are configured to independently produce first and second signals indicative of lactate concentrations measured at the first and second working electrodes, and
   wherein the sensor is configured to be partially inserted into the individual's skin;
b) applying a potential to the first working electrode and applying a potential to the second working electrode of the analyte sensor;
c) obtaining a first signal at or above an oxidation-reduction potential of the first lactate-responsive sensing area, the first signal being proportional to a concentration of lactate in the fluid;
d) obtaining a second signal at or above an oxidation-reduction potential of the second lactate-responsive sensing area, the second signal being proportional to a concentration of lactate in the fluid; and
e) correlating the first and second signals to first and second lactate concentrations in the fluid.

(20) The method of (19), further comprising determining a hybrid variance between the first and second lactate concentrations.

(21) The method of (20), wherein determining the hybrid variance comprises:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

(22) The method of (21), further comprising transmitting an average of the first and second lactate concentrations to be displayed if the hybrid variance is less than or equal to about 40%.

(23) The method of (21) or (22), further comprising detecting a noisy patch if a hybrid variance is greater than about 40% for about an hour or more.

(24) The method of claim (23), further comprising calculating an absolute relative difference (ARD) between standard deviations of the first and second lactate concentrations for a period of time and transmitting an average of the first and second lactate concentrations for the period of time to be displayed when the ARD is less than or equal to about 40%.

(25) The method of (24), further comprising calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(26) The method of any one of (19-25), wherein the first and second signals indicative of lactate concentrations are simultaneously measured at the first and second working electrodes.

(27) The method of any one of (19-26), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(28) The method of any one of (19-27), wherein continuously measuring signals indicative of lactate concentrations comprises measuring signals indicative of lactate concentrations about every minute.

(29) The method of any one of (19-28), wherein the first lactate-responsive sensing area comprises lactate oxidase.

(30) The method of any of (19-29), wherein the first lactate-responsive sensing area comprises a first polymer and a first electron transfer agent.

(31) The method of (30), wherein the first electron transfer agent is covalently bonded to the first polymer.

(32) The analyte sensor of (30) or (31), wherein the first electron transfer agent comprises an osmium complex.

(33) The method of any one of (30-32), wherein the first polymer is a poly(4-vinylpyridine)-based polymer.

(34) The method of any one of (30-33), wherein the first lactate-responsive sensing area further comprises a first crosslinker.

(35) The method of (34), wherein the first crosslinker is PEGDGE.

(36) The method of any one of (19-35), wherein the second lactate-responsive sensing area comprises lactate oxidase.

(37) The method of any one of (19-36), wherein the second lactate-responsive sensing area comprises a second polymer and a second electron transfer agent.

(38) The method of (37), wherein the second electron transfer agent is covalently bonded to the second polymer.

(39) The method of (37) or (38), wherein the second electron transfer agent comprises an osmium complex.

(40) The method of any one of (37-39), wherein the second polymer is a poly(4-vinylpyridine)-based polymer.

(41) The method of any one of (37-39), wherein the second lactate-responsive sensing area further comprises a second crosslinker.

(42) The method of (41), wherein the second crosslinker is PEGDGE.

(43) The method of any one of (19-42), wherein the sensor further comprises a reference electrode.

(44) The method of (19-43), wherein the sensor further comprises a counter electrode.

(45) The method of any one of (19-44), wherein the membrane comprises at least a crosslinked polyvinylpyridine homopolymer or copolymer.

(46) A method comprising:
a) continuously measuring signals indicative of lactate concentrations in a biological fluid with a sensing system comprising a lactate-responsive sensor, the lactate-responsive sensor comprising:
   a substrate;
   a first working electrode located on the substrate;
   a second working electrode located on the substrate;
   a first lactate-responsive sensing area disposed on a surface of the first working electrode;
   a second-responsive sensing area disposed on a surface of the second working electrode; and
   a membrane that is permeable to lactate overcoating the first and second lactate-responsive sensing areas,
   wherein the first and second lactate-responsive sensing areas are configured to independently produce first and second signals indicative of lactate concentrations measured at the first and second working electrodes, and
   wherein the sensor is configured to be partially inserted into a user's skin;
b) communicating first and second signals indicative of lactate concentrations measured at the first and second working electrodes to a processor;
c) correlating the first and second signals to corresponding first and second lactate concentrations; and
d) determining hybrid variance between the first and second lactate concentrations.

(47) The method of (46), wherein determining the hybrid variance comprises:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

(48) The method of (47), further comprising transmitting an average of the first and second lactate concentrations to be displayed if the hybrid variance is less than or equal to about 40%.

(49) The method of (47) or (48), further comprising detecting a noisy patch if a hybrid variance is greater than about 40% for about an hour or more.

(50) The method of claim (49), further comprising calculating an absolute relative difference (ARD) between standard deviations of the first and second lactate concentrations for a period of time and transmitting an average of the first and second lactate concentrations for the period of time to be displayed when the ARD is less than or equal to about 40%.

(51) The method of (50), further comprising calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(52) The method of any one of (46-51), wherein the first and second signals indicative of lactate concentrations are simultaneously measured at the first and second working electrodes.

(53) The method of any one of (46-52), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(54) The method of any one of (46-53), wherein continuously measuring signals indicative of lactate concentrations comprises measuring signals indicative of lactate concentrations about every minute.

(55) The method of any one of (46-54), wherein the first lactate-responsive sensing area comprises lactate oxidase.

(56) The method of any one of (46-55), wherein the first lactate-responsive sensing area comprises a first polymer and a first electron transfer agent.

(57) The method of (56), wherein the first electron transfer agent is covalently bonded to the first polymer.

(58) The method of (56) or (57), wherein the first electron transfer agent comprises an osmium complex.

(59) The method of any one of (56-58), wherein the first polymer is a poly(4-vinylpyridine)-based polymer.

(60) The method of any one of (56-59), wherein the first lactate-responsive sensing area further comprises a first crosslinker.

(61) The method of (60), wherein the first crosslinker is PEGDGE.

(62) The method of any one of (46-61), wherein the second lactate-responsive sensing area comprises lactate oxidase.

(63) The method of any one of (46-61), wherein the second lactate-responsive sensing area comprises a second polymer and a second electron transfer agent.

(64) The method of (63), wherein the second electron transfer agent is covalently bonded to the second polymer.

(65) The method of (63) or (64), wherein the second electron transfer agent comprises an osmium complex.

(66) The method of any one of (63-65), wherein the second polymer is a poly(4-vinylpyridine)-based polymer.

(67) The method of any one of (63-66), wherein the second lactate-responsive sensing area further comprises a second crosslinker.

(68) The method of (67), wherein the second crosslinker is PEGDGE.

(69) The method of any one of (46-68), wherein the sensor further comprises a reference electrode.

(70) The method of (46-69), wherein the sensor further comprises a counter electrode.

(71) The method of any one of (46-70), wherein the membrane comprises at least a crosslinked polyvinylpyridine homopolymer or copolymer.

(72) A sensor control device comprising the sensor of any one of (1-18) and a processor communicatively coupled to the sensor.

(73) The sensor control device of (72), wherein the sensor is configured to continuously measure signals indicative of lactate concentrations in the individual's biological fluid and communicate the signals indicative of lactate concentrations to the processor.

(74) The sensor control device of (73), wherein the processor is configured to correlate the first and second signals to corresponding first and second lactate concentrations and determine hybrid variance between the first and second lactate concentrations.

(75) The sensor control device of (74), wherein, to determine hybrid variance between the first and second lactate concentrations, the processor is configured to:
a) calculate the mean of the first lactate concentration and the second lactate concentration; and
b) when the mean is less than or equal to 2.5 mM lactate, calculate standard deviation of the first and second lactate concentrations and multiplying the standard deviation by 40 to obtain the hybrid variance; or
c) when the mean is greater than 2.5 mM lactate, calculating covariance between the first and second lactate concentrations and multiplying the covariance by 100 to obtain the hybrid variance.

(76) The sensor control device of (75), wherein the processor is configured to transmit an average of the first and second lactate concentrations to be displayed if the hybrid variance is less than or equal to about 40%.

(77) The sensor control device of (75) or (76), wherein the processor is configured to detect a noisy patch if a hybrid variance is greater than about 40% for about an hour or more.

(78) The sensor control device of (77), wherein the processor is configured to calculate an absolute relative difference (ARD) between standard deviations of the first and second lactate concentrations for a period of time and displaying an average of the first and second lactate concentrations for the period of time to be displayed when the ARD is less than or equal to about 40%.

(79) The sensor control device of (78), wherein the processor is configured to calculate the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(80) The sensor control device of any one of (72-79), wherein the sensor is configured to simultaneously measure the first and second signals indicative of lactate concentrations.

(81) The sensor control device of any one of (72-80), wherein the sensor is configured to continuously measure signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(82) The sensor control device of any one of (72-81), wherein the sensor is configured to continuously measure signals indicative of lactate concentrations about every minute.

(83) A sensing system comprising the sensor control device of any one of (72-82) and a reader device.

(84) An analyte sensor comprising:
a proximal portion configured to be positioned above a user's skin, and
a distal portion configured to be transcutaneously positioned through the user's skin, the distal portion comprising:
   a substrate;
   a first working electrode located on the substrate;
   a second working electrode located on the substrate;
   a first lactate-responsive sensing area disposed on a surface of the first working electrode;
   a second lactate-responsive sensing area disposed on a surface of the second working electrode; and
   a membrane that is permeable to lactate overcoating the first and second lactate-responsive sensing areas,
   wherein the first and second lactate-responsive sensing areas are configured to independently produce first and second signals indicative of lactate concentrations measured at the first and second working electrodes.

(85) The analyte sensor of (84), wherein the first lactate-responsive sensing area comprises lactate oxidase.

(86) The analyte sensor of (84) or (85), wherein the first lactate-responsive sensing area comprises a first polymer and a first electron transfer agent.

(87) The analyte sensor of (86), wherein the first electron transfer agent is covalently bonded to the first polymer.

(88) The analyte sensor of (87) or (87), wherein the first electron transfer agent comprises an osmium complex.

(89) The analyte sensor of any one of (86-88), wherein the first polymer is a poly(4-vinylpyridine)-based polymer.

(90) The analyte sensor of any one of (86-89), wherein the first lactate-responsive sensing area further comprises a first crosslinker.

(91) The analyte sensor of (90), wherein the first crosslinker is PEGDGE.

(92) The analyte sensor of any one of (84-91), wherein the second lactate-responsive sensing area comprises lactate oxidase.

(93) The analyte sensor of any one of (84-92), wherein the second lactate-responsive sensing area comprises a second polymer and a second electron transfer agent.

(94) The analyte sensor of (93), wherein the second electron transfer agent is covalently bonded to the second polymer.

(95) The analyte sensor of (93) or (94), wherein the second electron transfer agent comprises an osmium complex.

(96) The analyte sensor of any one of (93-95), wherein the second polymer is a poly(4-vinylpyridine)-based polymer.

(97) The analyte sensor of any one of (93-96), wherein the second lactate-responsive sensing area further comprises a second crosslinker.

(98) The analyte sensor of (97), wherein the second crosslinker is PEGDGE.

(99) The analyte sensor of any one of (84-98), wherein the sensor further comprises a reference electrode.

(100) The analyte sensor of (84-99), wherein the sensor further comprises a counter electrode.

(101) The analyte sensor of any one of (84-100), wherein the membrane comprises at least a crosslinked polyvinylpyridine homopolymer or copolymer.

(102) A method for monitoring lactate levels in an individual, comprising:
a) exposing an analyte sensor of a sensing system to a fluid; wherein the analyte sensor comprises:
   a proximal portion configured to be positioned above an individual's skin, and
   a distal portion configured to be transcutaneously positioned through the user's skin, the distal portion comprising:
      a substrate;
      a first working electrode located on the substrate;
      a second working electrode located on the substrate;
      a first lactate-responsive sensing area disposed on a surface of the first working electrode;
      a second lactate-responsive sensing area disposed on a surface of the second working electrode; and
      a membrane that is permeable to lactate overcoating the first and second lactate-responsive sensing areas,
      wherein the first and second lactate-responsive sensing areas are configured to independently produce first and second signals indicative of lactate concentrations measured at the first and second working electrodes;
b) applying a potential to the first working electrode and applying a potential to the second working electrode of the analyte sensor;
c) obtaining a first signal at or above an oxidation-reduction potential of the first lactate-responsive sensing area, the first signal being proportional to a concentration of lactate in the fluid;
d) obtaining a second signal at or above an oxidation-reduction potential of the second lactate-responsive sensing area, the second signal being proportional to a concentration of lactate in the fluid; and
e) correlating the first and second signals to first and second lactate concentrations in the fluid.

(103) The method of (102), further comprising determining a hybrid variance between the first and second lactate concentrations.

(104) The method of (103), wherein determining the hybrid variance comprises:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

(105) The method of (104), further comprising transmitting an average of the first and second lactate concentrations to be displayed if the hybrid variance is less than or equal to about 40%.

(106) The method of (104) or (105), further comprising detecting a noisy patch if a hybrid variance is greater than about 40% for about an hour or more.

(107) The method of claim (106), further comprising calculating an absolute relative difference (ARD) between standard deviations of the first and second lactate concentrations for a period of time and transmitting an average of the first and second lactate concentrations for the period of time to be displayed when the ARD is less than or equal to about 40%.

(108) The method of (107), further comprising calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(109) The method of any one of (102-108), wherein the first and second signals indicative of lactate concentrations are simultaneously measured at the first and second working electrodes.

(110) The method of any one of (102-109), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(111) The method of any one of (102-110), wherein continuously measuring signals indicative of lactate concentrations comprises measuring signals indicative of lactate concentrations about every minute.

(112) The method of any one of (102-111), wherein the first lactate-responsive sensing area comprises lactate oxidase.

(113) The method of any of (102-112), wherein the first lactate-responsive sensing area comprises a first polymer and a first electron transfer agent.

(114) The method of (113), wherein the first electron transfer agent is covalently bonded to the first polymer.

(115) The method of (113) or (114), wherein the first electron transfer agent comprises an osmium complex.

(116) The method of any one of (113-115), wherein the first polymer is a poly(4-vinylpyridine)-based polymer.

(117) The method of any one of (113-116), wherein the first lactate-responsive sensing area further comprises a first crosslinker.

(118) The method of any one of (113-117), wherein the first crosslinker is polyethylene glycol diglycidyl ether (PEGDGE).

(119) The method of any one of (102-118), wherein the second lactate-responsive sensing area comprises lactate oxidase.

(120) The method of any one of (102-119), wherein the second lactate-responsive sensing area comprises a second polymer and a second electron transfer agent.

(121) The method of (120), wherein the second electron transfer agent is covalently bonded to the second polymer.

(122) The method of (119) or (120), wherein the second electron transfer agent comprises an osmium complex.

(123) The method of any one of (120-122), wherein the second polymer is a poly(4-vinylpyridine)-based polymer.

(124) The method of any one of (120-123), wherein the second lactate-responsive sensing area further comprises a second crosslinker.

(125) The method of (124), wherein the second crosslinker is PEGDGE.

(126) The method of any one of (102-125), wherein the sensor further comprises a reference electrode.

(127) The method of any one of (102-126), wherein the sensor further comprises a counter electrode.

(128) The method of any one of (102-127), wherein the membrane comprises at least a crosslinked polyvinylpyridine homopolymer or copolymer.

(129) A method comprising:
a) continuously measuring signals indicative of lactate concentrations in a biological fluid with a sensing system comprising a lactate-responsive sensor, the lactate-responsive sensor comprising:
   a proximal portion configured to be positioned above a user's skin, and
   a distal portion configured to be transcutaneously positioned through the user's skin, the distal portion comprising:
      a substrate;
      a first working electrode located on the substrate;
      a second working electrode located on the substrate;
      a first lactate-responsive sensing area disposed on a surface of the first working electrode;
      a second-responsive sensing area disposed on a surface of the second working electrode; and
      a membrane that is permeable to lactate overcoating the first and second lactate-responsive sensing areas,
      wherein the first and second lactate-responsive sensing areas are configured to independently produce first and second signals indicative of lactate concentrations measured at the first and second working electrodes;
b) communicating first and second signals indicative of lactate concentrations measured at the first and second working electrodes to a processor;
c) correlating the first and second signals to corresponding first and second lactate concentrations; and
d) determining hybrid variance between the first and second lactate concentrations.

(130) The method of (129), wherein determining the hybrid variance comprises:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

(131) The method of (130), further comprising transmitting an average of the first and second lactate concentrations to be displayed if the hybrid variance is less than or equal to about 40%.

(132) The method of (130) or (131), further comprising detecting a noisy patch if a hybrid variance is greater than about 40% for about an hour or more.

(133) The method of claim (132), further comprising calculating an absolute relative difference (ARD) between standard deviations of the first and second lactate concentrations for a period of time and transmitting an average of the first and second lactate concentrations for the period of time to be displayed when the ARD is less than or equal to about 40%.

(134) The method of (133), further comprising calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(135) The method of any one of (129-134), wherein the first and second signals indicative of lactate concentrations are simultaneously measured at the first and second working electrodes.

(136) The method of any one of (129-135), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(137) The method of any one of (129-136), wherein continuously measuring signals indicative of lactate concentrations comprises measuring signals indicative of lactate concentrations about every minute.

(138) The method of any one of (139-137), wherein the first lactate-responsive sensing area comprises lactate oxidase.

(139) The method of any of (129-138), wherein the first lactate-responsive sensing area comprises a first polymer and a first electron transfer agent.

(140) The method of (139), wherein the first electron transfer agent is covalently bonded to the first polymer.

(141) The method of (139) or (140), wherein the first electron transfer agent comprises an osmium complex.

(142) The method of any one of (139-141), wherein the first polymer is a poly(4-vinylpyridine)-based polymer.

(143) The method of any one of (139-142), wherein the first lactate-responsive sensing area further comprises a first crosslinker.

(144) The method of (143), wherein the first crosslinker is PEGDGE.

(145) The method of any one of (129-144), wherein the second lactate-responsive sensing area comprises lactate oxidase.

(146) The method of any one of (129-145), wherein the second lactate-responsive sensing area comprises a second polymer and a second electron transfer agent.

(147) The method of (146), wherein the second electron transfer agent is covalently bonded to the second polymer.

(148) The method of (146) or (147), wherein the second electron transfer agent comprises an osmium complex.

(149) The method of any one of (146-148), wherein the second polymer is a poly(4-vinylpyridine)-based polymer.

(150) The method of any one of (146-149), wherein the second lactate-responsive sensing area further comprises a second crosslinker.

(151) The method of (150), wherein the second crosslinker is PEGDGE.

(152) The method of any one of (129-151), wherein the sensor further comprises a reference electrode.

(153) The method of any one of (129-152), wherein the sensor further comprises a counter electrode.

(154) The method of any one of (129-153), wherein the membrane comprises at least a crosslinked polyvinylpyridine homopolymer or copolymer.

(155) A sensor control device comprising the sensor of any one of (84-101) and a processor communicatively coupled to the sensor.

(156) The sensor control device of (155), wherein the sensor is configured to measure signals indicative of lactate concentrations in the individual's biological fluid and communicate the signals indicative of lactate concentrations to the processor.

(157) The sensor control device of (156), wherein the processor is configured to correlate the first and second signals to corresponding first and second lactate concentrations and determine hybrid variance between the first and second lactate concentrations.

(158) The sensor control device of (157), wherein, to determine hybrid variance between the first and second lactate concentrations, the processor is configured to:
a) calculate the mean of the first lactate concentration and the second lactate concentration; and
b) when the mean is less than or equal to 2.5 mM lactate, calculate standard deviation of the first and second lactate concentrations and multiplying the standard deviation by 40 to obtain the hybrid variance; or
c) when the mean is greater than 2.5 mM lactate, calculating covariance between the first and second lactate concentrations and multiplying the covariance by 100 to obtain the hybrid variance.

(159) The sensor control device of (158), wherein the processor is configured to transmit an average of the first and second lactate concentrations to be displayed if the hybrid variance is less than or equal to about 40%.

(160) The sensor control device of (158) or (159), wherein the processor is configured to detect a noisy patch if a hybrid variance is greater than about 40% for about an hour or more.

(161) The sensor control device of (160), wherein the processor is configured to calculate an absolute relative difference (ARD) between standard deviations of the first and second lactate concentrations for a period of time and transmitting an average of the first and second lactate concentrations for the period of time to be displayed when the ARD is less than or equal to about 40%.

(162) The sensor control device of (161), wherein the processor is configured to calculate the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.

(163) The sensor control device of any one of (155-162), wherein the sensor is configured to simultaneously measure the first and second signals indicative of lactate concentrations.

(164) The sensor control device of any one of (155-163), wherein the sensor is configured to continuously measure signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(165) The sensor control device of any one of (155-164), wherein the sensor is configured to continuously measure signals indicative of lactate concentrations about every minute.

(166) A sensing system comprising the sensor control device of any one of (155-165) and a reader device.

(167) An analyte sensor comprising:
a proximal portion configured to be positioned above a user's skin, and
a distal portion configured to be transcutaneously positioned through the user's skin, the distal portion comprising:
   a substrate;
   a first working electrode located on the substrate;
   a second working electrode located on the substrate;
   a first analyte-responsive sensing area disposed on a surface of the first working electrode;
   a second analyte-responsive sensing area disposed on a surface of the second working electrode; and
   a membrane that is permeable to analyte overcoating the first and second analyte-responsive sensing areas,
   wherein the first and second analyte-responsive sensing areas are configured to independently produce first and second signals indicative of analyte concentrations measured at the first and second working electrodes.

(168) The analyte sensor of (167), wherein the first analyte-responsive sensing area comprises a first analyte-responsive enzyme.

(169) The analyte sensor of (167) or (168), wherein the first analyte-responsive sensing area comprises a first polymer and a first electron transfer agent.

(170) The analyte sensor of (169), wherein the first electron transfer agent is covalently bonded to the first polymer.

(171) The analyte sensor of (169) or (170), wherein the first electron transfer agent comprises an osmium complex.

(172) The analyte sensor of any one of (169-171), wherein the first polymer is a poly(4-vinylpyridine)-based polymer.

(173) The analyte sensor of any one of (169-172), wherein the first analyte-responsive sensing area further comprises a first crosslinker.

(174) The analyte sensor of (173), wherein the first crosslinker is PEGDGE.

(175) The analyte sensor of any one of (167-174), wherein the second analyte-responsive sensing area comprises a second analyte-responsive enzyme.

(176) The analyte sensor of any one of (164-175), wherein the second analyte-responsive sensing area comprises a second polymer and a second electron transfer agent.

(177) The analyte sensor of (93), wherein the second electron transfer agent is covalently bonded to the second polymer.

(178) The analyte sensor of (93) or (94), wherein the second electron transfer agent comprises an osmium complex.

(179) The analyte sensor of any one of (93-95), wherein the second polymer is a poly(4-vinylpyridine)-based polymer.

(180) The analyte sensor of any one of (93-96), wherein the second analyte-responsive sensing area further comprises a second crosslinker.

(181) The analyte sensor of (97), wherein the second crosslinker is PEGDGE.

(182) The analyte sensor of any one of (84-98), wherein the sensor further comprises a reference electrode.

(183) The analyte sensor of (84-99), wherein the sensor further comprises a counter electrode.

(184) The analyte sensor of any one of (84-100), wherein the membrane comprises a polyvinylpyridine-co-styrene polymer.

(185) The analyte sensor of any one of (84-100), wherein the analyte is glucose.

(186) The analyte sensor of any one of (84-101), wherein the first analyte-responsive enzyme is glucose oxidase.

(187) The analyte sensor of any one of (84-102), wherein the second analyte-responsive enzyme is glucose oxidase.

(188) A method for monitoring analyte levels in an individual, comprising:
a) exposing the analyte sensor of any one of (167-187) to a fluid;
b) applying a potential to the first working electrode and applying a potential to the second working electrode of the analyte sensor;
c) obtaining a first signal at or above an oxidation-reduction potential of the first analyte-responsive sensing area, the first signal being proportional to a concentration of analyte in the fluid;
d) obtaining a second signal at or above an oxidation-reduction potential of the second analyte-responsive sensing area, the second signal being proportional to a concentration of analyte in the fluid; and
e) correlating the first and second signals to first and second analyte concentrations in the fluid by a processor.

(189) The method of (188), further comprising determining a hybrid variance between the first and second analyte concentrations by the processor.

(190) The method of (189), wherein determining the hybrid variance comprises:
i) calculating the mean of the first analyte concentration and the second analyte concentration; and
ii) when the mean is less than or equal to 2.5 mM analyte, calculating standard deviation of the first and second analyte concentrations and multiplying the standard deviation by 40 to obtain the hybrid variance; or
iii) when the mean is greater than 2.5 mM analyte, calculating covariance between the first and second analyte concentrations and multiplying the covariance by 100 to obtain the hybrid variance.

(191) The method of (190), further comprising transmitting an average of the first and second analyte concentrations to be displayed if the hybrid variance is less than or equal to about 40%.

(192) The method of (190) or (191), further comprising detecting a noisy patch if a hybrid variance is greater than about 40% for about an hour or more.

(193) The method of claim (192), further comprising calculating an absolute relative difference (ARD) between standard deviations of the first and second analyte concentrations for a period of time and transmitting an average of the first and second analyte concentrations for the period of time to be displayed when the ARD is less than or equal to about 40%.

(194) The method of (193), further comprising calculating the ARD between standard deviations of the first and second analyte concentrations for a period of time and transmitting the first analyte concentrations for the period of time to be displayed when the ARD is greater than or equal to about 40% and when the first analyte concentrations have a lower standard deviation than the second analyte concentrations; or transmitting the second analyte concentrations for the period of time to be displayed when the ARD is greater than or equal to about 40% and when the second analyte concentrations have a lower standard deviation than the first analyte concentrations.

(195) The method of any one of (188-194), wherein the first and second signals indicative of analyte concentrations are simultaneously measured at the first and second working electrodes.

(196) The method of any one of (188-195), wherein continuously measuring signals indicative of analyte concentrations comprises measuring signals indicative of analyte concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(197) The method of any one of (188-196), wherein continuously measuring signals indicative of analyte concentrations comprises measuring signals indicative of analyte concentrations about every minute.

(198) A method comprising:
a) continuously measuring signals indicative of analyte concentrations in a biological fluid with the analyte sensor of (167-187),
b) communicating first and second signals indicative of analyte concentrations measured at the first and second working electrodes to a processor;
c) correlating the first and second signals to corresponding first and second analyte concentrations by the processor; and
d) determining hybrid variance between the first and second analyte concentrations by the processor.

(199) The method of (198), wherein determining the hybrid variance comprises:
i) calculating the mean of the first analyte concentration and the second analyte concentration; and
ii) when the mean is less than or equal to 2.5 mM analyte, calculating standard deviation of the first and second analyte concentrations and multiplying the standard deviation by 40 to obtain the hybrid variance; or
iii) when the mean is greater than 2.5 mM analyte, calculating covariance between the first and second analyte concentrations and multiplying the covariance by 100 to obtain the hybrid variance.

(200) The method of (199), further comprising transmitting an average of the first and second analyte concentrations to be displayed if the hybrid variance is less than or equal to about 40%.

(201) The method of (199) or (200), further comprising detecting a noisy patch if a hybrid variance is greater than about 40% for about an hour or more.

(202) The method of claim (201), further comprising calculating an absolute relative difference (ARD) between standard deviations of the first and second analyte concentrations for a period of time and transmitting an average of the first and second analyte concentrations for the period of time to be displayed when the ARD is less than or equal to about 40%.

(203) The method of (202), further comprising calculating the ARD between standard deviations of the first and second analyte concentrations for a period of time and transmitting the first analyte concentrations for the period of time to be displayed when the ARD is greater than or equal to about 40% and when the first analyte concentrations have a lower standard deviation than the second analyte concentrations; or transmitting the second analyte concentrations for the period of time to be displayed when the ARD is greater than or equal to about 40% and when the second analyte concentrations have a lower standard deviation than the first analyte concentrations.

(204) The method of any one of (199-203), wherein the first and second signals indicative of analyte concentrations are simultaneously measured at the first and second working electrodes.

(205) The method of any one of (199-204), wherein continuously measuring signals indicative of analyte concentrations comprises measuring signals indicative of analyte concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(206) The method of any one of (199-205), wherein continuously measuring signals indicative of analyte concentrations comprises measuring signals indicative of analyte concentrations about every minute.

(207) A sensor control device comprising the sensor of any one of (167-187) and a processor communicatively coupled to the sensor.

(208) The sensor control device of (207), wherein the sensor is configured to measure signals indicative of analyte concentrations in the individual's biological fluid and communicate the signals indicative of analyte concentrations to the processor.

(209) The sensor control device of (208), wherein the processor is configured to correlate the first and second signals to corresponding first and second analyte concentrations and determine hybrid variance between the first and second analyte concentrations.

(210) The sensor control device of (209), wherein, to determine hybrid variance between the first and second analyte concentrations, the processor is configured to:
a) calculate the mean of the first analyte concentration and the second analyte concentration; and
b) when the mean is less than or equal to 2.5 mM analyte, calculate standard deviation of the first and second analyte concentrations and multiplying the standard deviation by 40 to obtain the hybrid variance; or
c) when the mean is greater than 2.5 mM analyte, calculating covariance between the first and second analyte concentrations and multiplying the covariance by 100 to obtain the hybrid variance.

(211) The sensor control device of (210), wherein the processor is configured to transmit an average of the first and second analyte concentrations to be displayed if the hybrid variance is less than or equal to about 40%.

(212) The sensor control device of (210) or (211), wherein the processor is configured to detect a noisy patch if a hybrid variance is greater than about 40% for about an hour or more.

(213) The sensor control device of (212), wherein the processor is configured to calculate an absolute relative difference (ARD) between standard deviations of the first and second analyte concentrations for a period of time and transmitting an average of the first and second analyte concentrations for the period of time to be displayed when the ARD is less than or equal to about 40%.

(214) The sensor control device of (213), wherein the processor is configured to calculate the ARD between standard deviations of the first and second analyte concentrations for a period of time and transmitting the first analyte concentrations for the period of time to be displayed when the ARD is greater than or equal to about 40% and when the first analyte concentrations have a lower standard deviation than the second analyte concentrations; or transmitting the second analyte concentrations for the period of time to be displayed when the ARD is greater than or equal to about 40% and when the second analyte concentrations have a lower standard deviation than the first analyte concentrations.

(215) The sensor control device of any one of (155-162), wherein the sensor is configured to simultaneously measure the first and second signals indicative of analyte concentrations.

(216) The sensor control device of any one of (207-215), wherein the sensor is configured to continuously measure signals indicative of analyte concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(217) The sensor control device of any one of (207-216), wherein the sensor is configured to continuously measure signals indicative of analyte concentrations about every minute.

(218) A sensing system comprising the sensor control device of any one of (207-217) and a reader device.

### EXAMPLES

The example presented below is provided for the purpose of illustration only and the embodiments described herein should in no way be construed as being limited to this example. Rather, the embodiments should be construed to encompass any and all variations which become evident as a result of the teaching provided herein.

### Example 1

A study of the variance between lactate concentrations independently measured by two channels in a sensor was conducted. The study included 18 subjects and each wore 4 sensors (2 on each arm) for one use cycle of 15 days. The dual lactate sensor design is shown in FIG. 6. 55 sensors had evaluable data of more than 2 days. The other 16 sensors had less than 2 days of data due to defects in the sensors (e.g., broken, crack, adhesive issue), or early sensor fall-off.

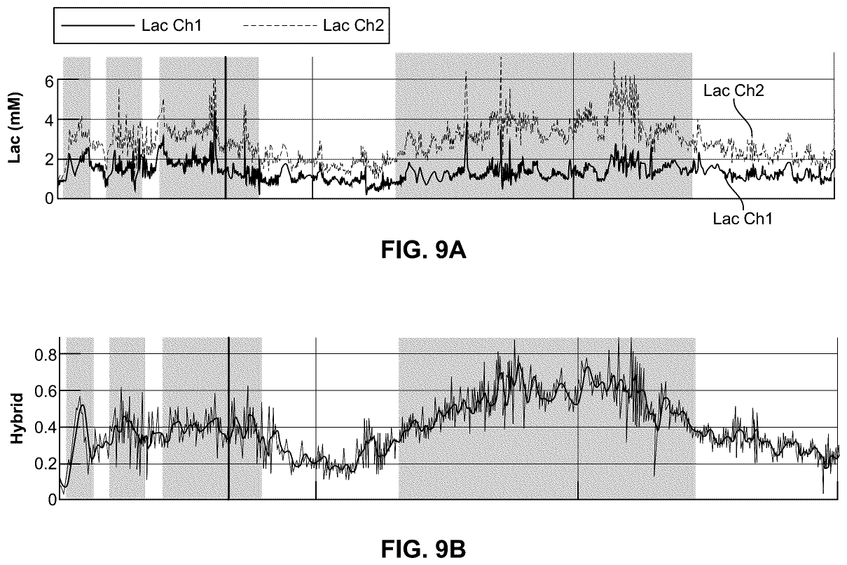

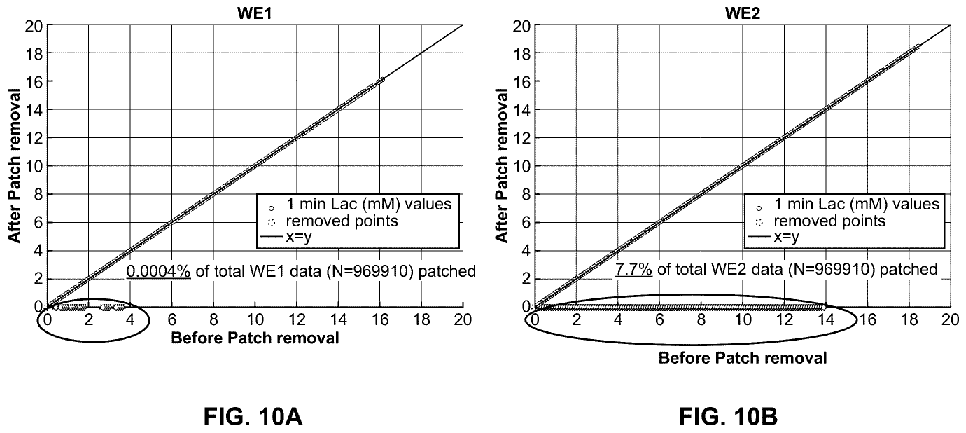

FIG. 8 shows the algorithm used to detect variance between measurements derived from the first and second working electrodes (first and second channels). The signal sensitivity from the first and second working electrodes were about the same (2.131 nA/mM for the first working electrode and 2.075 nA/mM for the second working electrode) such that it was not necessary to weight the measurements obtained from the sensors. FIG. 9A shows a graph of the lactate measurements derived from the first and second working electrodes for one of the sensors. FIG. 9B shows a graph of the hybrid variance between the simultaneous lactate measurements derived from the first and second working electrodes for the sensor in FIG. 9A. FIGS. 10A-B show graphs of lactate measurements derived from the first and second working electrodes before and after noisy segments were detected by the algorithm and removed. The noisy segments removed by the algorithm are circled and, as shown, more noisy segments were observed in the second working electrode.

### Example 2

Formulations for Lactate-Responsive Sensing areas Deposition:

Lactate oxidase was combined with an osmium-containing poly(4-vinylpyridine)-based polymer (Os-PVP) in aqueous solution formulations as specified in Tables 1 and 2 shown below.

TABLE 1
Formulation 1

| | in 10 mM MES | |
|---|---|---|
| | (pH 5.5) | Volume |
| Component | mg/mL | mL |
| Buffer | | |
| LOX | 80 | 0.32 |
| HSA | 80 | 0.32 |
| Os-PVP | 40 | 0.24 |
| PEGDGE400 | 40 | 0.16 |

TABLE 2
Formulation 2

| | in 10 mM MES | | Final |
|---|---|---|---|
| | (pH 5.5) | Volume | Concentration |
| Component | mg/mL | mL | mg/mL |
| Buffer | | | 10 mM |
| LOX | 130 | 0.32 | 40 |
| HSA | 60 | 0.32 | 18.5 |
| Os-PVP | 40 | 0.24 | 9.2 |
| PEGDGE400 | 40 | 0.16 | 6.2 |

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, can need to be revisited. Further, the Examiner is also reminded that any disclaimer made in the instant application should not be read into or against the parent application.

As used herein, the phrase “in some embodiments” in relation to a feature means that the feature may be present in any embodiment, unless the feature is obviously technically incompatible with that embodiment. Furthermore, any subset of features of one embodiment can be combined with any subset of features from any other embodiment(s) in any combination, unless such combination is obviously technically incompatible.

1. An electrochemical analyte sensor for detecting lactate in vivo, the sensor comprising:
a proximal portion configured to be positioned above a user's skin; and
a distal portion configured to be transcutaneously positioned through the user's skin such that the distal portion is in contact with the user's interstitial fluid to detect lactate in vivo; the distal portion comprising:
a substrate;
a first working electrode located on the substrate;
a second working electrode located on the substrate;
a first lactate-responsive sensing area disposed on a surface of the first working electrode;
a second lactate-responsive sensing area disposed on a surface of the second working electrode; and
a membrane that is permeable to lactate overcoating the first and second lactate-responsive sensing areas,
wherein the first and second lactate-responsive sensing areas are configured to independently produce first and second signals indicative of lactate concentrations measured at the first and second working electrodes.

2. The analyte sensor of claim 1, wherein the first lactate-responsive sensing area comprises lactate oxidase.

3. The analyte sensor of claim 1, wherein the first lactate-responsive sensing area comprises a first polymer and a first electron transfer agent.

4. The analyte sensor of claim 3, wherein the first electron transfer agent is covalently bonded to the first polymer.

5. The analyte sensor of claim 1, wherein the second lactate-responsive sensing area comprises lactate oxidase.

6. The analyte sensor of claim 1, wherein the second lactate-responsive sensing area comprises a second polymer and a second electron transfer agent.

7. The analyte sensor of claim 6, wherein the second electron transfer agent is covalently bonded to the second polymer.

8. The analyte sensor of claim 1, wherein the distal portion further comprises a reference electrode and a counter electrode.

9. The analyte sensor of claim 1, wherein the membrane comprises at least a crosslinked polyvinylpyridine homopolymer or copolymer.

10. A method for monitoring lactate levels in vivo in an individual, comprising:
a) exposing the analyte sensor of claim 1 to interstitial fluid;
b) applying a potential to the first working electrode and applying a potential to the second working electrode of the analyte sensor;
c) obtaining a first signal at or above an oxidation-reduction potential of the first lactate-responsive sensing area, the first signal being proportional to a concentration of lactate in the interstitial fluid;
d) obtaining a second signal at or above an oxidation-reduction potential of the second lactate-responsive sensing area, the second signal being proportional to a concentration of lactate in the interstitial fluid; and
e) correlating the first and second signals to first and second lactate concentrations in the interstitial fluid.

11. The method of claim 10, further comprising determining a variance between the first and second lactate concentrations.

12. The method of claim 10, wherein the first lactate-responsive sensing area comprises lactate oxidase.

13. The method of claim 10, wherein the first lactate-responsive sensing area comprises a first polymer and a first electron transfer agent.

14. The method of claim 13, wherein the first electron transfer agent is covalently bonded to the first polymer.

15. The method of claim 10, wherein the second lactate-responsive sensing area comprises lactate oxidase.

16. The method of claim 10, wherein the second lactate-responsive sensing area comprises a second polymer and a second electron transfer agent.

17. The method of claim 16, wherein the second electron transfer agent is covalently bonded to the second polymer.

18. The method of claim 10, wherein the distal portion further comprises a reference electrode and a counter electrode.

19. The method of claim 10, wherein the membrane comprises at least a crosslinked polyvinylpyridine homopolymer or copolymer.

20. A method, comprising:
a) continuously measuring signals indicative of lactate concentrations in a biological fluid with a sensing system comprising the sensor of claim 1;
b) communicating first and second signals indicative of lactate concentrations measured at the first and second working electrodes to a processor;
c) correlating the first and second signals to corresponding first and second lactate concentrations; and
d) determining hybrid variance between the first and second lactate concentrations.

21. The method of claim 20, wherein determining the hybrid variance comprises:
i) calculating the mean of the first lactate concentration and the second lactate concentration; and
ii) when the mean is less than or equal to 2.5 mM lactate, calculating standard deviation of the first and second lactate concentrations and multiplying the standard deviation by 40 to obtain the hybrid variance; or
iii) when the mean is greater than 2.5 mM lactate, calculating covariance between the first and second lactate concentrations and multiplying the covariance by 100 to obtain the hybrid variance.

22. The method of claim 21, further comprising transmitting an average of the first and second lactate concentrations to be displayed if the hybrid variance is less than or equal to about 40%.

23. The method of claim 21, further comprising detecting a noisy patch if a hybrid variance is greater than about 40% for about an hour or more.

24. The method of claim 23, further comprising calculating an absolute relative difference (ARD) between standard deviations of the first and second lactate concentrations for a period of time and transmitting an average of the first and second lactate concentrations for the period of time to be displayed when the ARD is less than or equal to about 40%.

25. The method of claim 24, further comprising calculating the ARD between standard deviations of the first and second lactate concentrations for a period of time and transmitting the first lactate concentrations for the period of time to be displayed when the ARD is greater than or equal to about 40% and when the first lactate concentrations have a lower standard deviation than the second lactate concentrations; or transmitting the second lactate concentrations for the period of time to be displayed when the ARD is greater than or equal to about 40% and when the second lactate concentrations have a lower standard deviation than the first lactate concentrations.
